(19) United States
(12) Patent Application Publication
(10) Pub. No.: US 2023/0337944 A1
(43) Pub. Date: Oct. 26, 2023

(54) SYSTEM AND DEVICE FOR QUANTIFYING MOTOR CONTROL DISORDER

(71) Applicants: David Joshua SZMULEWICZ, Port Melbourne (AU); THE FLOREY INSTITUTE OF NEUROSCIENCE AND MENTAL HEALTH, Parkville (AU); MURDOCH CHILDREN'S RESEARCH INSTITUTE, Parkville (AU); DEAKIN UNIVERSITY, Geelong (AU)

(72) Inventors: David Joshua SZMULEWICZ, Port Melbourne (AU); Pubudu Nishantha PATHIRANA, Williams Landing (AU); Ragil KRISHNA, Geelong West (AU); Louise Anne CORBEN, Parkville (AU); Malcolm Kenneth HORNE, Melbourne (AU)

(21) Appl. No.: 18/010,869

(22) PCT Filed: Jun. 18, 2021
(86) PCT No.: PCT/AU2021/050637

§ 371 Date: Dec. 16, 2022

(30) Foreign Application Priority Data
Jun. 19, 2020 (AU) 2020902051

(51) Int. Cl. A61B 5/11; A61B 5/00

(52) U.S. Cl. CPC A61B 5/1125; A61B 5/4842; A61B 5/7264; A61B 5/742

(57) ABSTRACT

A movement monitoring system for objectively quantifying a motor control disorder in a subject comprises a movement detection device generating movement data representing movement of a limb of the subject and an analyser for analysing the movement data. The movement detection device comprises sensors measuring at least motion of the device and pressure applied to the device by the subject. The analyser comprises a processor and a memory containing code which, when executed by the processor, receives the movement data generated by the movement detection device, applies the received movement data to an algorithmic model stored in the memory and identifies one or more features from the movement data that represent disordered movement by the subject, and calculates from the one or more identified features a score corresponding to the existence of the motor control disorder in the subject.

### TECHNICAL FIELD

The present disclosure relates to a system and device for monitoring movement for objectively quantifying a motor control disorder in a subject, as well as an automated method for doing the same. It relates particularly, but not exclusively, to a system, device and automated method for objectively quantifying a motor control disorder using movement detection device.

### BACKGROUND

Cerebellar Ataxia (CA or Ataxia) is a complex clinical term which carries two broad meanings. First, it is used to describe the abnormal movement that arises from dysfunction of the cerebellum or of its afferent paths. Thus, ataxia can result from strokes or Multiple sclerosis (MS) affecting the cerebellum, from neurodegenerations, from infections (e.g. post chicken pox), from toxins (alcohol), from many genetic disorders and from disorders of the afferent pathways (e.g. injury to the peripheral nerves or vestibular apparatus). There are also numerous episodic ataxias and there is an uncertain relationship with migraine. Second, ataxia appears in the specific name of specific disorders described in the 19th century —such as Friedreich's Ataxia and Spinocerebellar Ataxia.

Friedreich Ataxia (FA) is a life-shortening, progressive, autosomal recessive disorder. It is a neurodegenerative disease affecting the central and peripheral nervous systems, the heart, the pancreas and the musculoskeletal system. The neurological manifestations of FA include gait ataxia, loss of limb reflexes, dysarthria, reduction in proprioception and loss of vibrational sense. Other manifestations include cardiomyopathy, scoliosis, foot deformity and diabetes. Clinical features typically appear during adolescence (between 5 and 15 years), usually manifesting as gait ataxia and loss of coordination. A person with FA is wheelchair bound on average 15 years after symptom onset. Limb ataxia affects coordination and dexterity making it increasingly difficult to engage in basic daily activities such as dressing, drinking, eating and toileting known collectively as Activities of Daily Life (ADL). Difficulties with ADL may lead to embarrassment, social isolation and loss of independence and is an important index of the quality of life experienced by an individual with FA.

The overall prevalence of ataxia is not known. Prevalence of inherited ataxias is around 0.004-0.008%. The prevalence of other forms of ataxia (excluding alcohol) is not known but together are much more common, possibly 5 times more common.

Treatment is currently limited. Physiotherapy is commonly used, but with the exception of vestibular hypofunction (impaired function of the inner ear balance system) the evidence of benefit is limited. On the other hand, there is active research, with the possibility of imminent molecular therapies for the inherited ataxias.

The clinical characteristics of ataxia were described over 100 years ago by Gordon Holmes and still form the basis of current clinical examination which is the main means of assessing ataxia. Assessments involve observing a subject performing several specific tasks chosen because they emphasise ataxic movements. Accurate assessment requires experience and, as most doctors (even neurologists) have limited exposure to ataxia, many people with ataxia are initially misdiagnosed. This clinical examination has been codified by rating scales such as the Friedreich Ataxia Rating Scale (FARS), and the International Cooperative Scale for Rating Ataxia (ICARS). These rating scales have been criticised for their subjectivity. The Nine Hole Peg test (9HPT) and the Box and Block test (BBT) are two tests that measure upper limb function in individuals with FA. The 9HPT measures the time taken to place pegs in a pegboard and the BBT measures the number of blocks placed in sections of a box in 60 seconds. These tests address the subjectivity of the rating scales but are limited by ‘floor’ and ‘ceiling’ effects. Rehabilitation centres (mainly physiotherapists) also use devices such as the Balance Master and Gaitrite to assess balance and gait however these are costly and of limited value for diagnosis.

There have been studies that use sensors to measure movement with an objective of measuring and objectively quantifying movement disorders. Some have involved use of video cameras to capture upper limb functionality of children, and game based rehabilitation exercise have been developed to measure upper limb displacements in individuals with FA. However, none to date have provided a reliable alternative to clinical assessment methods.

It would be desirable to provide a means for providing a measure or assessment of ataxia which is objective and ideally, measures severity as well as existence of ataxia.

The discussion of the background is included herein including reference to documents, acts, materials, devices, articles and the like is included to explain the context of the present disclosure. This is not to be taken as an admission or a suggestion that any of the material referred to was published, known or part of the common general knowledge in Australia or in any other country as at the priority date of any of the claims.

### SUMMARY OF INVENTION

A criticism of current clinical tests for determining presence or severity of ataxia is that they are subjective and so clinical assessments can vary between clinicians, even experienced clinicians and neurologists. Thus, an aspect of the disclosure is an instrumented method for assessment that involves a movement detection device for objective monitoring of the subject's movement.

Viewed from one aspect, the present disclosure provides a movement monitoring system for objectively quantifying a motor control disorder in a subject, the system comprising: (a) a movement detection device generating movement data representing movement of a limb of the subject, wherein the movement detection device comprises sensors measuring at least motion of the device and pressure applied to the device by the subject; and (b) an analyser for analysing the movement data. The analyser comprises a processor and a memory containing code which, when executed by the processor: (i) receives the movement data generated by the movement detection device; (ii) applies the received movement data to an algorithmic model stored in the memory and identifies one or more features from the movement data that represent disordered movement by the subject; and (ii) calculates from the one or more identified features a score corresponding to the existence of the motor control disorder in the subject.

Preferably the movement detection device generates movement data representing movement of an upper limb of the subject. The movement detection device may be e.g. body worn or may be attached to or comprise part of an object of daily living and ideally is compressed during movement a task performed by a subject.

In some embodiments, the analyser applies the received movement data to one or more of: (a) a first algorithmic model to identify a first set of features used by the processor to calculate a selection score which is indicative of presence or absence of the motor control disorder in the subject; (b) a second algorithmic model to identify a second set of features used by the processor to calculate a severity score which is indicative of severity of the motor control disorder in the subject; and (c) a third algorithmic model to identify a third set of features used by the processor to calculate a progression score which is indicative of progression of the motor control disorder in the subject. Typically, the first set of features and the second set of features are not identical, although some features may be common to both sets and one feature set may be a subset of the other feature set.

In some embodiments, the severity score calculated by the processor corresponds to a score obtained according to a clinical scale. For example, the severity score may correspond to a score that would otherwise be determined by a clinician performing assessment of the subject using assessment scales such as modified-FARS (mFARS), FARS, 9HPT, BBT, Scale for the Assessment and Rating of Ataxia (SARA), ICARS, ADL and the like.

In some embodiments, e.g. where the motor control disorder is ataxia, the first set of features used to calculate a selection score may comprise a single feature, ideally a pressure feature such as resonant frequency of pressure (Pr_{RF}). In other embodiments, the first set of features comprises some or all of the features Pr_{RF}, resonant frequency of acceleration in the x-axis (A_{CC}_{_{RF}}^{X}), magnitude at resonance of absolute angular velocity in the XYZ-axis (Gyr_{MR}^{XYZ}), movement smoothness using dimensionless jerk (Sₘ), accommodation time (Aₜ), and mean value of pressure (Pr_{M}). In a preferred embodiment, the feature set comprising resonant frequency of pitch angle using complimentary filter (θ_{RF}^{c}), Acc_{RF}^{X}, Sₘ, Pr_{RF}, Aₜ, Euclidean distance of the stabilisation phase (S_{T}), Pr_{M}, resonant frequency of roll angle using complimentary filter (Ø_{RF}^{c}) and Gyr_{MR}^{XYZ} provides a selection score yielding high identification of FA (99%).

In some embodiments, e.g. where the motor control disorder is ataxia, the second set of features used to calculate a severity score may comprise a single feature, or a plurality of or all the features selected from the group comprising: Pr_{RF}Pr_{M}, Aₜ and θ_{RF}^{c}. In some embodiments, the feature set comprising Pr_{M}, Aₜ and θ_{RF}^{c} is preferred.

In some embodiments, e.g. where the motor control disorder is ataxia, the third set of features used to calculate a progression score may comprise a single feature or a plurality of or all the features selected from a group comprising: MRₚᵣ, SRF_{gyr}, MSE_{IMF}_{₂}^{acc}, Sᵥ₁-HT_{gyr}, ROM_{θ}, MRᵥₑₗ, SRF_{acc}, Sᵥ₁-HTₚᵣ. In some embodiments, the feature set MRₚᵣ, SRF_{gyr}, MSE_{IMF}_{₂}^{acc}, Sᵥ₁-HT_{gyr} is preferred.

In some embodiments, e.g. when the motor control disorder is spasticity, the set of features used to indicate presence of spasticity may comprise one or more or all of Standard deviation value of Pressure (Pr_{SD}), RMS value of Pressure (Prᵣₘₛ), magnitude at resonance of pressure (Pr_{MR}) and Pr_{RF}.

In some embodiments, the analyser categorises movement dysfunction in the subject by the processor calculating a contribution made by each of the first, second or third set of features to each of a plurality of movement characteristics that are attributable to movement dysfunction in the subject. The set of movement characteristics may correlate to clinically accepted descriptions of movement disorder. Such descriptions may relate, in some embodiments, to e.g. stability, timing, accuracy and rhythmicity of the movement. In some embodiments, the analyser sums the contribution made by each of the features to each of the plurality of movement characteristics to determine a collective contribution to each of the plurality of movement characteristics.

In some embodiments, the movement detection device comprises sensors measuring one or more of (a) position of the limb; (b) acceleration of the limb; and (c) angular position of the limb. In some embodiments, the movement detection device RECTIFIED SHEET (RULE 91) ISA/AU simulates or is incorporated into an object of daily living such as a cup, spoon, brush or the like and comprises one or more of: (a) a pressure sensor, (b) an accelerometer, and (c) a gyroscope.

Another criticism of current clinical tests is that they lack functional relevance to the daily activities which are perceived as important for people with ataxia or measures salient for rehabilitation. An important component of daily life for a person with FA is the capacity to drink a beverage independently. This requires the capacity to reach, grasp, transport and release a cup in a continuum of motion in preparation for drinking the contents of a canister or container. These subcomponents of the task are also reflected in other daily living tasks. Ensuring individuals with FA can continue to independently administer liquids may be an important achievement of therapeutic interventions.

Thus in some embodiments, the movement detection device comprises a canister with a grasping portion and a pressure sensor for measuring pressure applied to the grasping portion by the subject.

Preferably the movement monitoring system is configured to measure the motor control disorder during a movement task.

The movement monitoring system may be utilised to quantify a range of motor control disorders such as, but not limited to ataxia (in its various forms) and spasticity.

Viewed from another aspect, the present disclosure provides a movement detection device for use with a system for objectively quantifying motor control disorder in a subject, the movement detection device comprising: (a) a grasping portion; and (b) a movement sensor comprising at least a pressure sensor generating pressure data representing pressure applied to the grasping portion; wherein the movement detection device simulates or is incorporated into an object of daily living.

The object of daily living may be selected from a group including but not limited to a cup or drinking vessel, a spoon or eating utensil, brush, comb or the like.

The movement sensor typically comprises one or both of an accelerometer and a gyroscope generating motion data representing movement of the device in multiple axes.

In some embodiments, the movement detection device comprises a microcontroller receiving movement data from the motion sensor and optionally, a wireless communication module for wireless transmission of the movement data from the microcontroller to a receiving device. The receiving device may be any suitable device such as, for example, a smart phone, tablet, laptop, desktop computer, or dedicated device receiving the movement data from the motion sensor.

In some embodiments, the movement detection device comprises a canister simulating a cup or drinking vessel, the canister comprising a flexible body portion forming a fluid filled chamber and defining the grasping portion. In some embodiments, the pressure sensor is a differential pressure sensor with a first input in fluid communication with the chamber and a second input in fluid communication with atmospheric pressure. A one-way valve may be provided for releasable coupling with a fluid source to restore fluid pressure in the chamber.

In some embodiments, the canister comprises a rigid base containing a microcontroller and ideally, a wireless communication module.

The movement detection device is typically provided use with the movement monitoring system according to embodiments disclosed herein.

Viewed from another aspect, the present disclosure provides an automated method for objectively quantifying a motor control disorder in a subject, comprising the steps of: (a) receiving at a processor movement data corresponding to movements of a limb of the subject, the movement data comprising at least pressure data and motion data; (b) the processor applying the received movement data to an algorithmic model and identifying one or more features from the movement data that represent disordered movement in the subject; (c) the processor calculating, from the one or more identified features, a score quantifying the motor control disorder in the subject; and (d) the processor generating a display signal causing the calculated score to be presented on a display device.

In some embodiments, the processor applies the received movement data to one or more of: (a) a first algorithmic model to identify a first set of features used by the processor to calculate a selection score which is indicative of presence or absence of the motor control disorder in the subject; (b) a second algorithmic model to identify a second set of features used by the processor to calculate a severity score which is indicative of severity of the motor control disorder in the subject; and (c) a third algorithmic model to identify a third set of features used by the processor to calculate a progression score which is indicative of progression of the motor control disorder in the subject.

It is to be understood that the one or more features identified by the automated method correspond with the features as disclosed previously in relation to the earlier described aspect relating to a system for objectively quantifying a motor control disorder in a subject.

In some embodiments, the method further comprises categorising movement dysfunction in the subject, by the processor calculating a contribution made by each of the first or second set of features to each of a plurality of movement characteristics that are attributable to movement dysfunction in the subject. In some embodiments, the plurality of movement characteristics correlate to clinically accepted descriptions of movement disorder or aspects of movement disorder such as stability, timing, accuracy and rhythmicity.

In some embodiments, the received movement data is obtained from a movement detection device and comprises at least one or both of: pressure data corresponding to pressure applied to the device by the subject; and motion data comprising one or more of position of the limb, acceleration of the limb and angular position of the limb. Ideally, the received movement data is collected while the subject performs a movement task and preferably wherein the movement task is or simulates an activity of daily living. In some embodiments, the motor control disorder may include e.g. ataxia (in its various forms) and/or spasticity.

Viewed from another aspect, the present disclosure provides an automated method for determining an algorithmic model for use in a system or method for objectively quantifying a motor control disorder in a subject comprising the steps of an algorithm designing processor: (a) receiving movement data from a plurality of training subjects who had undergone clinical assessment for the motor control disorder; the movement data comprising pressure data, acceleration data and angular velocity data obtained from a movement detection device while each of the training subjects performed a movement exercise; (b) receiving for each of the plurality of training subjects clinical scores corresponding to the clinical assessment; (c) extracting from the received movement data a plurality of features representing aspects of movement during the movement exercise; (d) selecting from the plurality of features a subset of features using a feature selection algorithm; and (e) using a machine learning approach to build the algorithmic model.

In some embodiments, the feature selection algorithm is Neighbourhood Component Analysis with regularization (NCA-R) although other feature selection algorithms may be utilised.

In one embodiment, the algorithmic model is a first algorithmic model used to calculate a selection score which is indicative of presence or absence of the motor control disorder in the subject, and the machine learning approach is a k-Nearest Neighbour (k-NN) classification model. In some embodiments, the subset of features identified by the feature selection algorithm for the first algorithmic model is selected from the group of subsets comprising: (a) Pr_{RF}; (b) Pr_{RF}, A_{CC}_{_{RF}}^{X}, Gyro_{MR}^{XYZ}, Sₘ, Aₜ and Pr_{M}; and (c) θ_{RF}^{c}, Acc_{RF}^{X}, Sₘ, Pr_{RF}, Aₜ, S_{T}, Pr_{M}, θ_{RF}^{c} and Gyr_{MR}^{XYZ}.

In another embodiment, the algorithmic model is a second algorithmic model used to calculate a severity score which is indicative of severity of the motor control disorder in the subject, and the machine learning approach is a random-forest regression model (RFR). In some embodiments, the calculated selection and severity scores may be scaled to correspond to an accepted clinical scale such as FARS, mFARS, 9HPT, BBT, SARA, ICARS, ADL or the like, or some other clinical measure. In some embodiments, the subset of features identified by the feature selection algorithm for the second algorithmic model is selected from a group of comprising Pr_{RF} Pr_{M}, Aₜ and θ_{RF}^{c} and preferably comprises the feature set Pr_{M}, Aₜ and θ_{RF}^{c}.

In some embodiments, the pressure data is processed by the algorithm designing processor to identify a compressed phase and an uncompressed phase in each instance of the movement exercise.

In another embodiment, the algorithmic model is a third algorithmic model used to calculate a progression score which is indicative of progression of the motor control disorder in the subject between an initial and a subsequent clinical assessment during which movement data was received, and the feature selection algorithm comprises (i) extracting features of the movement data in one or more of time domain, frequency domain and time-frequency domain; (ii) identifying in the extracted features, relevant features showing a change in value at T2 relative to T1; and (iii) selecting from the relevant features, significant features which show a statistically significant change in value from T1 to T2. The statistical significance in the change in value may be assessed by a Wilcoxon signed-rank test for matched pairs. In some embodiments, the machine learning approach comprises Principal Component Analysis followed by linear regression, optionally with an assumption that the motor control disorder increases in severity between the initial and subsequent assessments. In some embodiments, the subset of features identified by the feature selection algorithm for the third algorithmic model is selected from a group of features comprising MRₚᵣ, SRF_{gyr}, MSE_{IMF}_{₂}^{acc}, Sᵥ₁-HT_{gyr}, ROM_{θ}, MRᵥₑₗ, SRF_{acc}, Sᵥ₁-HTₚᵣ; and preferably comprises MRₚᵣ, SRF_{gyr}, MSE_{IMF}_{₂}^{acc}, Sᵥ₁-HT_{gyr}.

Viewed from another aspect, the present disclosure provides a movement monitoring system for objectively quantifying a motor control disorder in a subject, the system comprising: (a) a movement detection device generating movement data representing movement of a limb of the subject; and (b) an analyser for analysing the movement data. The analyser comprises a processor and a memory containing code which, when executed by the processor: (i) receives the movement data generated by the movement detection device; (ii) applies the received movement data to an algorithmic model stored in the memory and identifies one or more features from the movement data that represent disordered movement by the subject; and (ii) calculates from the one or more identified features a score corresponding to the existence of the motor control disorder in the subject.

In another aspect, the present disclosure provides a movement detection device for use in objectively quantifying a motor control disorder, the movement detection device comprising a plurality of sensors measuring at least motion of the device and pressure applied to the device by the subject, wherein the device represents an object of daily living such as a cup or drinking vessel, a spoon or eating utensil, a brush or comb or the like. Preferably the device has a grasping portion and a pressure sensor of the device generates pressure data representing pressure applied to the gasping portion. Ideally, the device includes one or both of an accelerometer and a gyroscope generating motion data representing movement of the device in multiple axes. The device may also include a wireless communication module for wireless transmission of the movement data from the microcontroller to a receiving device which may be used to process the movement data.

In another aspect, the present disclosure provides for use of the systems, methods, and devices disclosed herein in evaluation, e.g. in a clinical trial, of a therapy for treating a motor control disorder, such as ataxia. Therapies may include medicines or physical therapies, and treatment may involve one or more of mitigating symptoms or reducing or eliminating disease.

It is to be noted that any one of the aspects mentioned above may include any of the features of any of the other aspects mentioned above and may include any of the features of any of the embodiments described below, as appropriate.

It is to be understood each of the various aspects described herein may incorporate features, modifications and alternatives described in the context of one or more other aspects, such as but not limited to the features of the movement monitoring system, the movement detection device, and the automated method for objectively quantifying a motor control disorder in a subject. For efficiency, such features, modifications and alternatives have not been repetitiously disclosed for each and every aspect although one of skill in the art will appreciate that such combinations of features, modifications and alternatives disclosed for some aspects apply similarly for other aspects and are within the scope of and form part of the subject matter of this disclosure.

### BRIEF DESCRIPTION OF DRAWINGS

The present disclosure will now be described in greater detail with reference to the accompanying drawings. It is to be understood that the embodiments shown are examples only and are not to be taken as limiting the scope of the disclosure as defined in the claims appended hereto.

FIG. 1 is a schematic illustration of a movement monitoring system for objectively quantifying a motor control disorder in a subject.

FIG. 2A is a schematic illustration showing a front view of a movement detection device 200 according to an embodiment of the disclosure. FIG. 2B is a sectional view of the device in FIG. 2A.

FIG. 3 illustrates phases of movement involved in the movement task of preparing to drink from a cup or canister.

FIG. 4 is a schematic illustration exemplifying an approach to methodology for developing algorithmic models according to embodiments of the disclosure.

FIG. 5 illustrates a changing point algorithm to detect compressed and uncompressed phases of the movement task.

FIG. 6 depicts the difference between individuals with FA and controls in direct measures (acceleration (Acc), angular velocity (Gyr) and pressure (Pr)) and derived measures (velocity (Vel) and Euler angle (Euler)) in terms of mean and standard deviation obtained according to embodiments of the disclosure.

FIG. 7 is a box plot representation of separation obtained using pressure and movement features.

FIG. 8 is a chart showing weights obtained for (a) NCA_{RC} subset (b) NCA_{RR} subset using NCA-R selection.

FIG. 9 (a) shows separation (selection) achieved for KNN model using NCA_{RC} subset; (b) shows S.T.A.R. domain contribution using NCA_{RC}; (c) shows S.T.A.R. domain contribution using NCA_{RR} subset.

FIG. 9 is a schematic illustration showing steps in an automated method for objectively quantifying a motor control disorder in a subject, according to an embodiment of the disclosure.

FIG. 10 is a schematic illustration of an automated method for objectively quantifying a motor control disorder in a subject according to an embodiment of the disclosure.

FIG. 11 is a schematic illustration exemplifying an approach to feature extraction for obtaining a progression score.

FIG. 12 is a graph showing the magnitude at resonance for pressure (MRₚᵣ).

FIG. 13 shows graphically the difference between T1 and T2 values of resonant frequency (RF) and Second Resonant Frequency (SRF) of (a) acceleration, (b) angular velocity, and (c) pressure.

FIG. 14 shows graphically the Mean Square Energy (MSE) of the second Intrinsic Mode Function (IMF₂) obtained from acceleration data from all subjects.

FIGS. 15A and 15B shows graphically the first ten singular values obtained using the SvHT approach for the three measures acceleration, angular velocity and pressure (acc, gyr, pr).

FIG. 16(a) shows the features whose values at T2 had significantly changed from their values at T1. FIG. 16(b) shows the percentage change in feature values at T2 for the four features MRₚᵣ, SRF_{gyr}, MSE_{IMF}_{₂}^{acc} and Sᵥ₁-HT_{gyr}.

FIG. 17(a) shows graphically a comparison of clinical scores against the AIM-C score for progression in terms of longitudinal progression. FIG. 17(b) shows graphically the comparison in terms of percentage change.

FIG. 18 is a graphical representation showing correlation of features identified for use in calculating a progression score with clinical scores of GAA1, GAA2, Staging, ADL, NeuroUL and mFARS.

### DETAILED DESCRIPTION

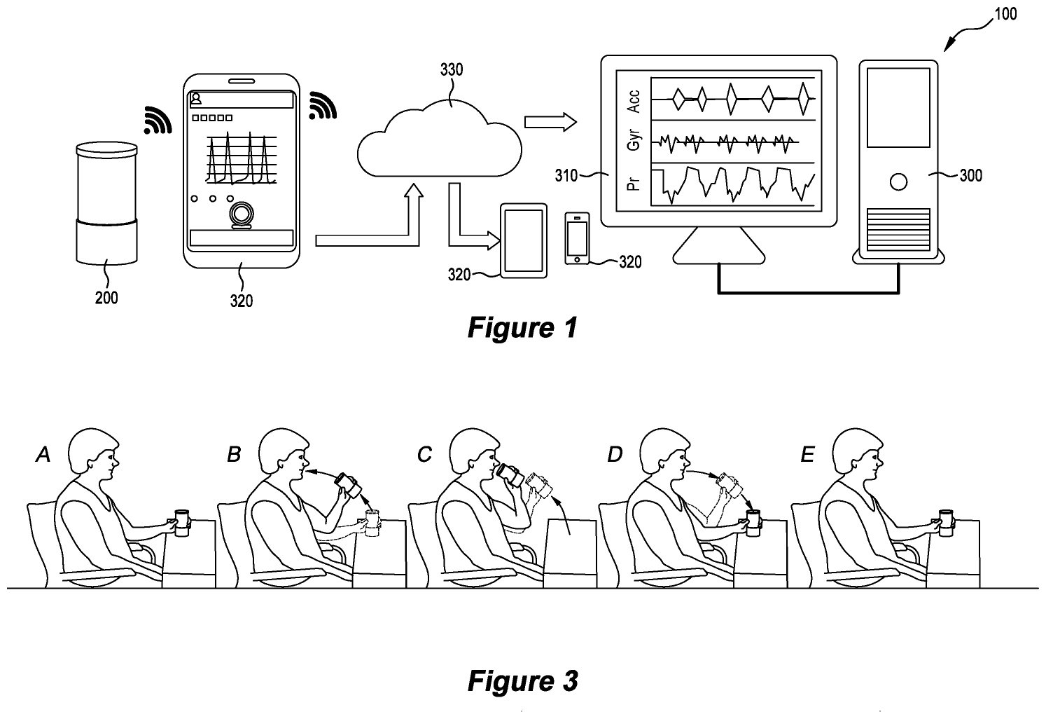

Referring firstly to FIG. 1, there is shown a movement monitoring system 100 for objectively quantifying a motor control disorder in a subject. System 100 comprises a movement detection device 200 generating movement data representing movement of a limb of the subject. Movement detection device 200 comprises sensors measuring at least movement of the device and pressure applied to the device by the subject. An analyser 300 analyses the movement data. Analyser 300 comprises a processor and a memory containing code which, when executed by the processor: (i) receives the movement data generated by the movement detection device; (ii) applies the received movement data to an algorithmic model stored in the memory and identifies one or more features from the movement data that represent disordered movement by the subject; and (ii) calculates from the one or more identified features a score corresponding to the existence of the motor control disorder in the subject.

The embodiment illustrated in FIG. 1 shows analyser 300 as a server or desktop computing device coupled with a display device 310 and in communication with receiving device 320 via communication network 330. However, it is to be understood that the functionality of analyser 300 may be provided in a variety of ways. For example, the functionality of analyser 300 may be local to or even incorporated into movement detection device 200 or receiving device 320. However, since functionality of analyser 300 is typically achieved in a software application, it is in some embodiments preferred that the processor of analyser 300 is remote from movement detection device 200 and indeed accessible e.g. in the “cloud” by communication network 330 so that it may process movement data from several or many movement detection devices 200 used to assess motor disorder in several or many subjects. Such subjects may be undergoing assessment at a single site, or at several different sites, each of which transmits movement data collected by the individual movement detection devices 200 to the analyser 300 for processing. Analyser 300 comprises a processor and memory components (not explicitly shown) which may be provided together in a single physical device or distributed across a plurality of physical devices that are communicatively coupled via a wireless or wired network, as would be understood by one of skill in the art.

In some embodiments, movement detection device 200 contains a communications interface, such as a Wi-Fi interface, which provides for wireless real time transmission of movement data collected by movement detection device 200 to a receiving device 320 such as a smart phone or tablet operated by an operator such as the subject or a clinician responsible for the subject's assessment. Receiving device 320 operates as a gateway for transmission of the movement data to analyser 300 for application of the received motion data to a first algorithmic model to identify a first set of features that may be used by a processor of the analyser 300 to calculate a selection score which is indicative of presence or absence of the motor control disorder in the subject. The selection score may be used e.g. in a clinical setting to diagnose a subject presenting with motor disorder symptoms. In some embodiments, the selection score confirms the presence or absence of ataxia or spasticity in the subject.

Alternatively/additionally, movement data received by analyser 300 may be applied to a second algorithmic model to identify a second set of features that may be used by a processor of the analyser to calculate a severity score which is indicative of severity of the motor control disorder in the subject. The severity score may be used in clinical settings to objectively assess the severity of motor disorder symptoms. In preferred embodiments, kinematic (pressure) and kinetic (motion) features extracted from the motion data are used, together with the algorithmic models, to produce scores, such as severity scores, that correlate with clinical scales such as modified-FARS, the Activities of Daily Living (ADL) scale, BBT and 9HPT. Severity scores from objective assessments repeated according to embodiments of the disclosure over time may be plotted or compared to evaluate progression or improvement in motor control disorder symptoms and be used to guide or evaluate therapies and rehabilitation.

Alternatively/additionally, movement data received by analyser 300 may be applied to a third algorithmic model to identify a third set of features that may be used by a processor of the analyser to calculate a progression score which is indicative of extent or rate of progression of disease and may be positive (i.e. advancement of disease) or negative (i.e. improvement e.g. with therapy). The progression score may be used in clinical settings to objectively assess progression of disease and/or effectiveness of therapy and/or to titrate therapy such that e.g. dosage increases are matched to a corresponding rate of progression. The progression score may alternatively/additionally be used in research settings or e.g. in clinical trials to evaluate effectiveness of novel therapies.

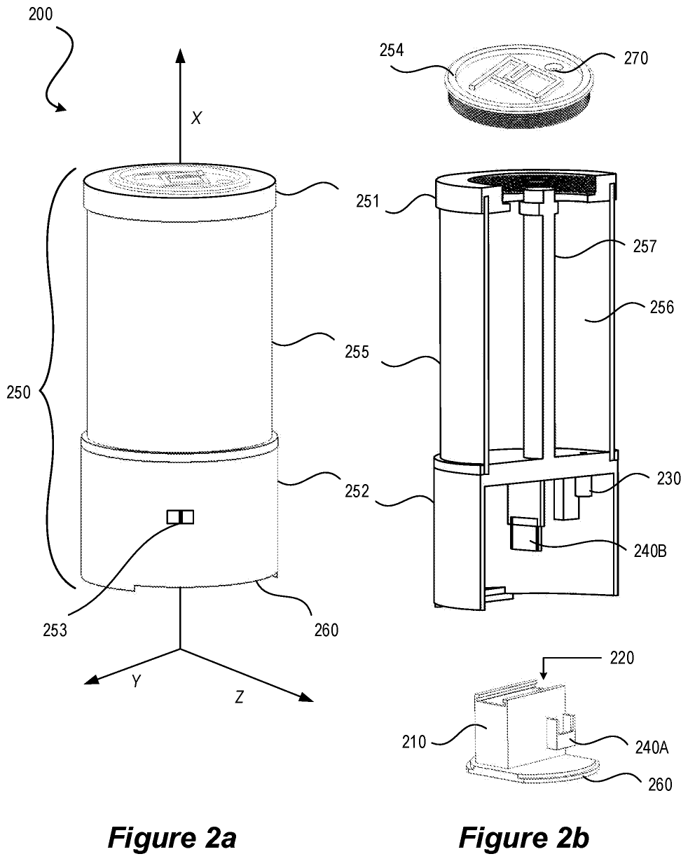

FIGS. 2A and 2B are schematic illustrations showing front and sectional views, respectively, of a movement detection device 200 according to an embodiment of the disclosure. Movement detection device 200 ideally simulates or is incorporated into an object of daily living such as a cup or drinking vessel, spoon, knife, fork or comb, and typically comprises a pressure sensor and a motion sensor comprising an accelerometer and a gyroscope. In the example shown, the object of daily living is a cup and the device is a canister having a main body 250 and a base unit 260.

In some embodiments, base unit 260 contains most electronic components of the device 200 including the motion sensor which may comprise a 9-axis inertial movement unit (IMU) 210 (e.g. Invensense MPU9250) interfaced with a micro-controller unit 220 (e.g. 32-bit ARM R Cortex-M3) having a Wi-Fi interface (e.g. GS2011), and a rechargeable battery (not shown) equipped with a power management circuit (not shown).

IMU 210 may comprise a triaxial accelerometer and a triaxial gyroscope with a range of +/−2000 degrees per second and optionally, a triaxial geomagnetic sensor. The accelerometer provides information about accelerations in all three directions and the gyroscope provides information about rotations around each axis. To mitigate small errors that build up in each axis over time causing drift in the absolute direction, algorithms utilised by a microprocessor in IMU 210 use the extra magnetic field information from the geomagnetic sensor to compensate for small drifts. Using IMU 210, inertial data is captured as changes in acceleration (±8 g) and angular velocity (±2000/s).

Connector 240A provides for releasable coupling of base unit 260 with a corresponding connector 240B on main body 250 using a friction fit, screw fit or other means. It is to be understood connectors 240A,B as shown are merely examples and that other means for connecting main body 250 and base unit 260 would be known to one of skill in the art.

Main body 250 has rigid cap 251, flexible body 255 defining chamber 256 and rigid wall portion 252 providing on/off switch 253. Rigid cap 251 has sealing lid 254 with air inlet 270, consisting of a one-way valve which permits air to be injected into the chamber 256 to restore the chamber's air pressure should there be leakage over time. Support structure 257 extends through chamber 256 to impart structural integrity to the canister device 200 although it is to be understood that a plurality of support structures or other means could be provided to impart the requisite structural integrity of the canister whilst also achieving the required flexibility and compressibility of flexible body 255.

Flexible body 255 provides a grasping portion for grasping by the subject and defines an air filled chamber 256. Pressure sensor 230 (e.g. MS4525DO DS5AI001DP, TE connectivity) is incorporated into main body 250 with one input port in fluid communication with air chamber 256 and the other port exposed to atmospheric pressure. Pressure sensor 230 transmits the 16 bit differential pressure data as an electrical signal to the micro-controller 220 e.g. via an I2C communication interface or similar. The differential pressure data, being the difference between atmospheric pressure and the varying pressure inside the flexible chamber 256 is transmitted to micro-controller 220. Both kinetic and kinematic data are transferred to receiving device 320 by Wi-Fi transmission.

Movement detection device 200 in this example is provided in the form of a canister of compact size and light weight (e.g. 185 grams) which is comparable to a cup or other drinking vessel typically used by the subject in daily living. In one example, canister device 200 may be manufactured using a multi-material additive manufacturing technique (Objet350 Connex3 3D printer, Stratasys Ltd., USA) wherein inkjet deposited droplets of photopolymer controlled at the minimal length scales 40 μm×80 μm×30 μm×35 μm are UV cured. The soft and hard surfaces of the composite may be printed respectively using VeroCyan (RGD841, shore hardness 85) and Agilus (transparent, shore hardness 30, flexible). The volume of the chamber 256 defined by flexible body 255 is, in the illustrated embodiment, 2.27×10⁷ mm³ (13.8 cubic in) and the pressure of air inside is maintained at −0.05 psi.

For assessment, a subject 300 is asked to sit at a table with forearms pronated and wrists in a natural position resting in front of them with hips and knees flexed to 90° and feet flat on the floor. The device is placed on the table in front of the subject and the subject is instructed to mimic the task of preparing to drink a glass of water using their dominant arm. The phases of movement involved in this preparatory task are illustrated in FIG. 3 and include A) extending their dominant arm to grasp the device (‘accommodating’ phase), B) transporting the device up towards their mouth until it just touches their lower lip (‘transporting’ phase), C) releasing from the transporting phase, D) returning the device back to the original position (‘releasing’ phase) and E) releasing the device back onto the table (‘stabilising’ phase). Typically, subjects are requested to repeat the preparatory task five times with approximately 5 seconds interval between each cycle to allow for mishaps (such as dropping the device) which can occur from time to time. However it is understood that the task may be performed fewer times, or even only once if the task is performed without mishaps.

Data collected by movement detection device 200 is transmitted wirelessly to receiving device 320 and forwards it on to analyser 300 where it is processed according to methods disclosed herein. It is to be understood, however, that transmission via receiving device 320 need not be wireless and the electronics unit of base unit 260 may be connected by a cable or other connector to the receiving device. In other embodiments, movement detection device 200 contains a SIM card or other means for communicating directly with analyser 300, omitting the intervening step of movement data transmission via receiving device 320. Analyser 300, processes received movement data for the subject according to embodiments disclosed herein, to determine a score such as a selection score or a severity score. In some embodiments the analyser 300 processes received movement data for the subject to determine scores for categories of movement dysfunction in the subject by a processor associated with the analyser calculating a contribution made by each of the first or second set of features to each of a plurality of movement characteristics that are attributable to movement dysfunction in the subject.

### Example 1—Selection Score and Severity Score

42 participants consisting of 20 controls (11 males, 9 females) with an average age of 35.8±13.43 years and twenty two individuals with FA of varying severities (12 males and 10 females) with an average age of 37.05±12.23 years took part in this study. All subjects performed a trial in which upper limb movement data was recorded using the movement detection device 200 described as an Ataxia Instrumented Measure-Canister (AIM-C).

All the participants were assessed using 9HPT and BBT and using clinical scales. All subjects performed a trial in which upper limb movement data was recorded using the movement detection device 200. Individuals with FA also underwent assessment using known clinical scales. The presence of spasticity in the wrist and long finger flexors was identified by clinical testing.

### Signal Pre-Processing

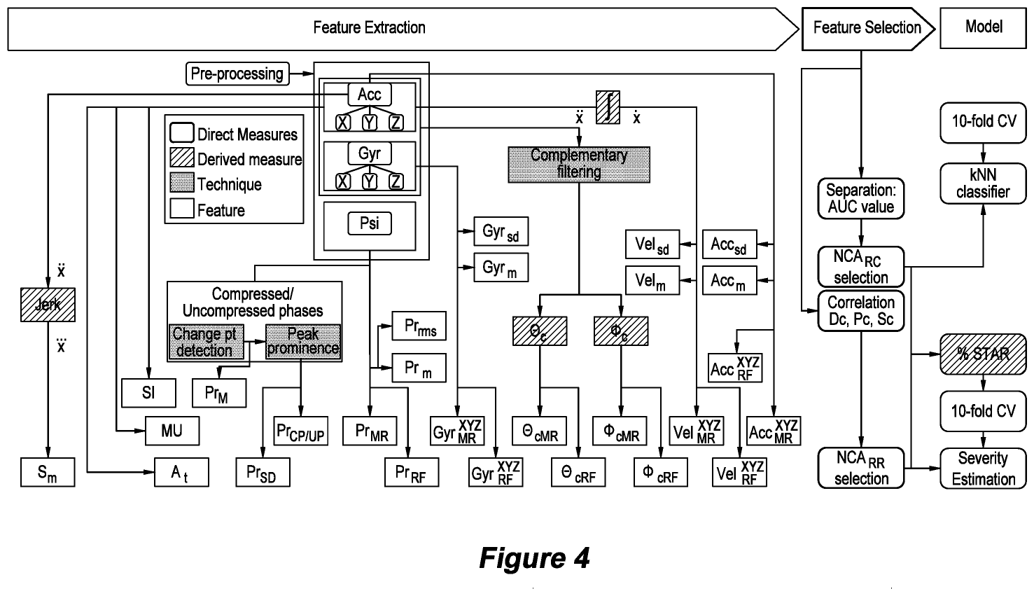

The rotational, translational and pressure data were sampled at 100 Hz and subsequently low pass filtered with a cut-off frequency of 20 Hz. A median filter was used to suppress noise transients in the data. A 6th order Savitzky-Golay filter smoothed data and contributed to minimising the drift effects with averaging. The functional architecture of the overall methodology is given in FIG. 4.

### Feature Extraction

IMU 210 measures acceleration and angular velocity from which the velocity and position were derived using known techniques (FIG. 4). Time domain features of pressure and motion data were initially considered in terms of statistical parameters such as mean and standard deviation. Blackman-Harris windowing technique was engaged to reduce spectral leakages to improve the algorithmic performance in the frequency domain analysis when capturing features such as magnitude at resonance (MR), resonant frequency (RF) and bandwidth (BW) in all pressure and motion measures. Features of further interest were extracted using various signal processing techniques as discussed at A) to D) below.

A) Complimentary filtering to estimate roll and pitch angles: The Euler angles related to angular measurements uncover distinct movement characteristics intrinsically linked to ataxia. Euler angles were obtained through complimentary filtering techniques while negating the contribution from the gravitational force. The sensor orientation was observed in terms of Euler angle (Roll φ, Pitch θ) variations in the preliminary analysis. The complimentary filter designed to estimate roll \hat{ϕ}ₐ_{_{c}} and pitch \hat{θ}ₐ_{_{c}} using accelerometer data is given as,

$${\hat{ϕ}}_{{a}_{c}} = {tan}^{- 1} ( \frac{{Acc}_{y}}{\sqrt{( {Acc}_{x}^{2} + {Acc}_{z}^{2} )}} ) \qquad ( 1 a )$$

$${\hat{θ}}_{{a}_{c}} = {tan}^{- 1} ( \frac{- {Acc}_{x}}{\sqrt{( {Acc}_{y}^{2} + {Acc}_{z}^{2} )}} ) \qquad ( 1 b )$$

With the initial conditions for roll \hat{ϕ}=ϕg_{co} and pitch \hat{θ}=θ_{d}_{_{gc}}, values obtained using gyroscope data with sample period dt=0.01 is given as,

\hat{ϕ}_{g}_{_{c}}=\hat{ϕ}+dt*(p+sin(\hat{ϕ})*tan(\hat{θ})*q+cos(\hat{ϕ})*tan(\hat{θ})*r) (2a)

\hat{ϕ}_{g}_{_{c}}=\hat{θ}+dt*(cos(\hat{ϕ})*q−sin(\hat{ϕ})*r) (2b)

the complimentary filter can be stated as,

\hat{ϕ}_{c}=(1−ω)*\hat{ϕ}_{g}_{_{c}}+ω*ϕₐ_{_{c}} (3a)

\hat{θ}_{c}=(1−ω)*\hat{θ}_{g}_{_{c}}+ω*\hat{θ}ₐ_{_{c}} (3b)

where p, q and rare the gyro readings in X, Y and Z axis respectively with the time constant ω. The complimentary filtering technique with ω=0.15 performed better than Madgwick's algorithm and Kalman filtering techniques. Table 1 describes features extracted in this Example.

B) Dimensionless jerk: The stability of the performance of the task was assessed by finding the smoothness Sₘ, which is log dimensionless jerk where the jerk is defined as the rate of change of acceleration in time t and is given as:

$${S}_{m} = ln ( \frac{{T}_{p}^{5}}{{L}_{m}^{2}} {\int }_{t 1}^{t 2} {{x}^{...} ( t )}^{2} dt ) \qquad ( 4 )$$

where [symbol] (t) is the jerk, t1 and t2 are initial and final times, Lₘ denotes the maximum peak velocity where Lₘ=max_{∀t∈[t1,t2]} \dot{x}(t) and Tₚ is the time period.

TABLE 1

| Sensor | Symbol | Feature description | Type | p-value | AUC | Power |
|---|---|---|---|---|---|---|
| IMU | Accₘ | Mean of absolute acceleration | Kinematic | 0.0589 | 0.8254 | 0.8353 |
| | Acc_{sd} | Standard deviation of abs. | | 0.0001 | 0.9019 | 0.8972 |
| | | acceleration | | | | |
| | Acc_{RF}^{X} | Resonant frequency of | | 0.0000 | 0.9999 | 0.9998 |
| | | acceleration in X-axis | | | | |
| | Acc_{MR}^{X} | Magnitude at resonance of | | 0.0735 | 0.9115 | 0.9050 |
| | | acceleration in X-axis | | | | |
| | Acc_{RF}^{Y} | Resonant frequency of | | 0.0000 | 0.9976 | 0.9998 |
| | | acceleration in Y-axis | | | | |
| | Acc_{MR}^{X} | Magnitude at resonance of | | 0.0535 | 0.5215 | 0.0117 |
| | | acceleration in Y-axis | | | | |
| | Acc_{RF}^{Z} | Resonant frequency of | | 0.0000 | 0.9379 | 0.9998 |
| | | acceleration in Z-axis | | | | |
| | Acc_{MR}^{Z} | Magnitude at resonance of | | 0.0001 | 0.6579 | 0.1430 |
| | | acceleration in Z-axis | | | | |
| | Acc_{RF}^{XYZ} | Resonant Frequency of abs. | | 0.0000 | 0.9876 | 0.9998 |
| | | acceleration in XYZ-axis | | | | |
| | | combined | | | | |
| | Acc_{MR}^{XYZ} | Magnitude at resonance of abs. | | 0.0001 | 0.8804 | 0.7643 |
| | | acceleration in XYZ-axis | | | | |
| | | combined | | | | |
| | Gyrₘ | Mean of absolute angular | | 0.0000 | 0.9330 | 0.1621 |
| | | velocity | | | | |
| | Gyr_{sd} | Standard deviation of abs. | | 0.0010 | 0.9617 | 0.0082 |
| | | angular velocity | | | | |
| | Gyr_{RF}^{X} | Resonant frequency of angular | | 0.0001 | 0.6077 | 0.3778 |
| | | velocity in X-axis | | | | |
| | Gyr_{MR}^{X} | Magnitude at resonance of | | 0.0014 | 0.6220 | 0.5727 |
| | | angular velocity in X-axis | | | | |
| | Gyr_{RF}^{Y} | Resonant frequency of angular | | 0.0000 | 0.8230 | 0.4889 |
| | | velocity in Y-axis | | | | |
| | Gyr_{MR}^{Y} | Magnitude at resonance of | | 0.0201 | 0.9282 | 0.7839 |
| | | angular velocity in Y-axis | | | | |
| | Gyr_{RF}^{Z} | Resonant frequency of angular | | 0.0000 | 0.8278 | 0.3644 |
| | | velocity in Z-axis | | | | |
| | Gyr_{MR}^{Z} | Magnitude at resonance of | | 0.0008 | 0.7990 | 0.8288 |
| | | angular velocity in Z-axis | | | | |
| | Gyr_{RF}^{XYZ} | Resonant frequency of abs. | | 0.0000 | 0.9641 | 0.9998 |
| | | angular velocity in XYZ-axis | | | | |
| | Gyr_{MR}^{XYZ} | Magnitude at resonance of abs. | | 0.0000 | 0.9856 | 0.9998 |
| | | angular velocity in XYZ-axis | | | | |
| | Velₘ | Mean value of absolute velocity | Derived | 0.0013 | 0.5818 | 0.3568 |
| | Vel_{sd} | Standard deviation of absolute | | 0.0082 | 0.6301 | 0.2766 |
| | | velocity | | | | |
| | Vel_{RF}^{X} | Resonant frequency of velocity | | 0.0000 | 0.8947 | 0.9772 |
| | | in X-axis | | | | |
| | Vel_{MR}^{X} | Magnitude at resonance of | | 0.0027 | 0.9633 | 0.6279 |
| | | velocity in X-axis | | | | |
| | Vel_{MR}^{Y} | Resonant frequency of angular | | 0.0000 | 0.9221 | 0.7285 |
| | | in Y-axis | | | | |
| | Vel_{RF}^{Y} | Magnitude at resonance of | | 0.0191 | 0.6665 | 0.6130 |
| | | velocity in Y-axis | | | | |
| | Vel_{RF}^{Z} | Resonant frequency of velocity | | 0.0000 | 0.8254 | 0.6666 |
| | | in Z-axis | | | | |
| | Vel_{MR}^{Z} | Magnitude at resonance of | | 0.0000 | 0.7440 | 0.7146 |
| | | velocity in Z-axis | | | | |
| | ∅_{c}^{RF} | Resonant frequency of roll | | 0.0000 | 0.9641 | 0.9998 |
| | | angle using complimentary | | | | |
| | | filter | | | | |
| | ∅_{MR}^{c} | Magnitude at resonance of roll | | 0.0000 | 0.6340 | 0.2827 |
| | | angle using complimentary | | | | |
| | | filter | | | | |
| | θ_{RF}^{c} | Resonant frequency of pitch | | 0.0000 | 0.9928 | 0.9998 |
| | | angle using complimentary | | | | |
| | | filter | | | | |
| | θ_{MR}^{c} | Magnitude at resonance of pitch | | 0.0000 | 0.7967 | 0.5234 |
| | | angle using complimentary | | | | |
| | | filter | | | | |
| | ∅_{RF}^{k} | Resonant frequency of roll | | 0.0000 | 0.6507 | 0.3923 |
| | | angle using kalman filter* | | | | |
| | ∅_{MR}^{k} | Magnitude at resonance of roll | | 0.0008 | 0.6244 | 0.0019 |
| | | angle using kalman filter* | | | | |
| | θ_{RF}^{k} | Resonant Frequency of pitch | | 0.0000 | 0.9920 | 0.9998 |
| | | angle using kalman filter* | | | | |
| | θ_{MR}^{k} | Magnitude at resonance of pitch | | 0.0001 | 0.8014 | 0.5941 |
| | | angle using kalman filter* | | | | |
| | ∅_{RF}^{m} | Resonant frequency of roll | | 0.0000 | 0.8913 | 0.8316 |
| | | angle using Madgwick's | | | | |
| | | algorithm* | | | | |
| | ∅_{MR}^{m} | Magnitude at resonance of roll | | 0.0080 | 0.5813 | 0.0117 |
| | | angle using Madgwick's | | | | |
| | | algorithm* | | | | |
| | θ_{RF}^{m} | Resonant frequency of pitch | | 0.0002 | 0.7795 | 0.8314 |
| | | angle using Madgwick's | | | | |
| | | algorithm* | | | | |
| | θ_{MR}^{m} | Magnitude at resonance of pitch | | 0.0060 | 0.8058 | 0.2020 |
| | | angle using Madgwick's | | | | |
| | | algorithm* | | | | |
| | Eulerₘ | Mean value of Euler angle | | 0.0010 | 0.6029 | 0.6378 |
| | Euler_{sd} | Standard deviation of Euler | | 0.0233 | 0.7402 | 0.3156 |
| | | angles | | | | |
| | Aₜ | Accomodation Time | | 0.0001 | 0.9565 | 0.7166 |
| | SI | Straightness Index** | | 0.0233 | 0.9027 | 0.3156 |
| | MU | Movement Units** | | 0.0040 | 0.8895 | 0.6675 |
| | S_{T} | Eucliean distance of | | 0.0005 | 0.9904 | 0.9651 |
| | | stabilisation phase | | | | |
| | Sₘ | Movement smoothness using | | 0.0006 | 0.9976 | 0.8311 |
| | | dimensionless jerk (dominant | | | | |
| | | direction) | | | | |
| Pressure | Prₘ | Mean value of Pressure | Kinetic | 0.0009 | 0.6124 | 0.0782 |
| | Prₛ | Standard deviation value of | | 0.0508 | 0.8130 | 0.8615 |
| | | Pressure | | | | |
| | Prᵣₘₛ | RMS value of Pressure | | 0.0059 | 0.7990 | 0.8515 |
| | Pr_{RF} | Resonant frequency of Pressure | | 0.0000 | 0.9999 | 0.8415 |
| | Pr_{MR} | Magnitude at resonance of | | 0.0050 | 0.5837 | 0.7885 |
| | | Pressure | | | | |
| | Pr_{BW} | FFT Bandwidth Power 3DB | | 0.0015 | 0.5502 | 0.7229 |
| | Pr_{SD} | Pressure variation during | | 0.0000 | 0.5359 | 0.6175 |
| | | compress cycle | | | | |
| | Pr_{CP/UP} | Assymetry Ratio | | 0.0050 | 0.6388 | 0.1516 |
| | Pr_{M} | Mean time period per pressure | | 0.0000 | 0.9833 | 0.9998 |
| | | cycle | | | | |
| Combined | SFS^{c} | Combination of features | All | 0.0000 | 0.9999 | 0.9999 |
| | | selected using NCA-R | | | | |
| | NCA_{RC} | Combination of features | | 0.0000 | 0.9999 | 0.9999 |
| | | selected using SFS | | | | |
| | SD^{c} | Combination of features | | 0.0000 | 0.9064 | 0.9999 |
| | | selected using SD | | | | |
| | RSFS^{c} | Combination of features | | 0.0000 | 0.9220 | 0.9999 |
| | | selected using RSFS | | | | |
| | Pr^{c} | Combination of kinetic features | | 0.0000 | 0.9999 | 0.9999 |
| | | selected | | | | |
| | AG^{c} | Combination of kinematic | | 0.0000 | 0.9999 | 0.9876 |
| | | features selected | | | | |

*Features utilised for comparison with features extracted in this study
**Features obtained based on existing studies of upper limb functionality

C) Phase of movement-based features: Four distinct phases of movement were identified during the movement task, namely: ‘grasping’, ‘transporting’, ‘releasing’ and ‘stabilising’ phases (FIG. 3) and specific features of the movement data were identified during these phases:
Straightness index (SI): the ratio of distance between the initial and final position and the real distance travelled, and
Movement units (MU): the difference between maximum velocity and a minimum value of velocity greater than a predetermined threshold (40 mm/s).

Euclidean distance and time duration of ‘accommodating’ and ‘stabilising’ phases (FIG. 3) were also captured as features.

D) Pressure data measures: The pressure data revealed fluctuations in the pressure applied to the flexible body 255 of the device 200, which can be considered as two distinct phases within a cycle:
Compressed phase (CP): The duration of this phase is the time the flexible body 255 of the device 200 is being deformed by the subject's grip. Its magnitude is the difference between the current pressure and the initial pressure rating (i.e. −0.05 psi).
Uncompressed phase (UP): The duration of this phase is the time that the surface deformation is negligible and the current pressure approaches the initial rating.

The change in phase was detected using a known change-point detection algorithm (R. Killick, P. Fearnhead, and I. A. Eckley, “Optimal detection of changepoints with a linear computational cost,” Journal of the American Statistical Association, vol. 107, no. 500, pp. 1590-1598, 2012). Accordingly Mₐ^{b} is the mean of a given time series of data S(i)ᵢ₌ₐⁱ=b, the residual or cost functional (J) associated with the changing point algorithm for the data series S(i) is defined as:

J(k)=Σᵢ₌₁^{k-1}(s(i)−M₁^{k})²+Σⱼ₌ₖⁿ(s(j)−Mₖⁿ)² (5)

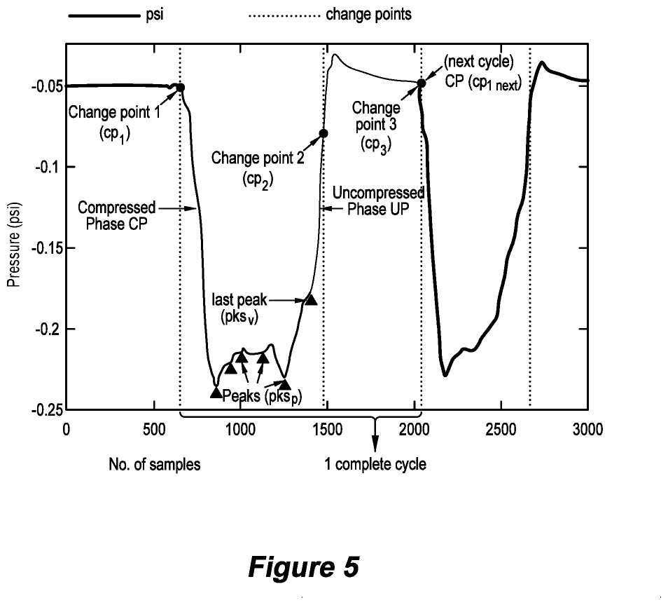

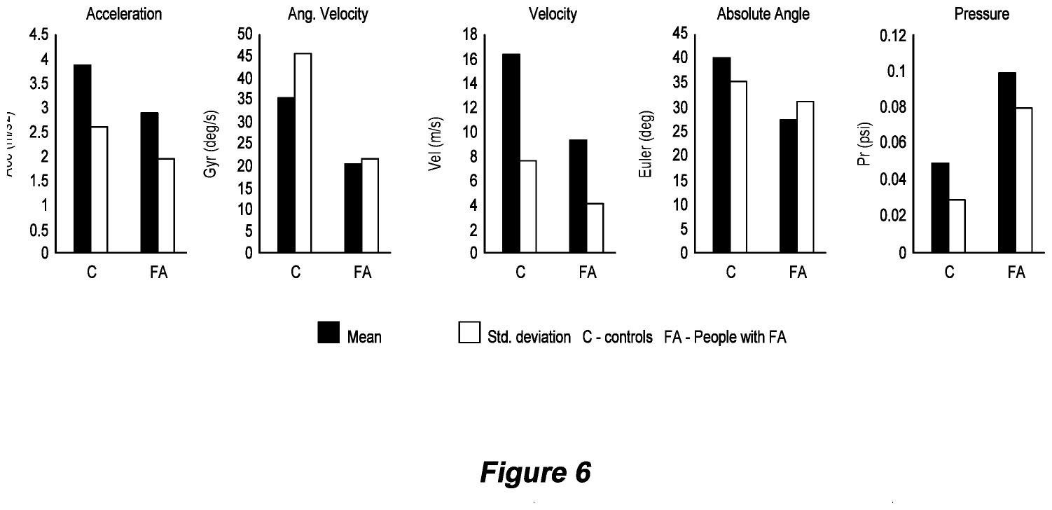

FIG. 5 illustrates a changing point algorithm to detect compressed and uncompressed phases of the movement task. The figure shows two pressure cycles, where the first cycle (1 complete cycle) illustrates CP and UP, ending at pksᵥ and cp₃ respectively. The change points 1 to 3 were detected via sequentially seeking min J(k), ∀k∈[1, . . . , n] to determine the start (cp₁) and end (cp₃) of a cycle as depicted in FIG. 5. The pressure spikes/peaks (pks) within a cycle were detected using peak prominence constraint [J. Griffié, L. Boelen, G. Burn, A. P. Cope, and D. M. Owen, “Topographic prominence as a method for cluster identification in single-molecule localisation data,” Journal of biophotonics, vol. 8, no. 11-12, pp. 925-934, 2015] after inverting the pressure signal and reducing the noise with a 6th order Savitzky-Golay filter. The peaks were detected with a prominence and a minimum peak height of 0.005 and 0.1 respectively and, as per our observation, the last peak denotes the transition from CP phase to UP phase.

The standard deviation in the amplitude of the pks (Pr_{SD}) was a feature pertaining to the CP phase. Asymmetry ratio feature (Pr_{CP/UP}) is the ratio of the duration of compressed CP and un-compressed pressure UP phases. Also, the average time duration of the complete cycle (Pr_{M}), i.e. time duration between cp₁ and cp₃ was considered as a feature of interest.

### Feature Selection

Of the features described in Table 1, only those that (i) effectively ‘separated’ the individuals with FA from controls and (i) showed correlation with clinical scales indicating ‘severity’ are described.

Separation: The feature separation for the two cohorts was quantified using Area Under the Curve AUC measure from the Receiver Operating Characteristics (ROC) which discriminates between data from the two cohorts using trapezoidal approximation. The AUC value also indicates the extent to which the two cohorts are classified within the estimated confident bounds.

Correlation: The movement and pressure data features were correlated with the commonly used ataxia scales and with scores for spasticity using three measures: Distance correlation D_{c}, Pearson correlation P_{c}, and Spearman rank correlation S_{c}. This was done so that the features with best correlation (combined/uncombined) could be identified and to establish the type of correlation.

Statistical analysis: Wilcoxon-Mann-Whitney signed-rank test was used to determine if the features extracted for the cohorts were statistically significant. G*Power (version 3.1.9.4) software with significance level (p-value) 0.01 and AUC>0.75 was used to determine whether the features held sufficient statistical-power (α) for the study (min. threshold: α≥80%, the effect-size for our sample size (42) is 1.06).

Feature selection techniques: Most of the extracted features could separate controls from individuals with FA with good statistical power (Table 1). To avoid the risk of over estimations when a large number of features are combined, feature selection algorithms were used to select the feature-sets that best estimated severity while separating between controls and individuals with FA. Four feature section algorithms; Sequential Forward Selection (SFS), Random Subset Feature Selection (RSFS), Statistical Dependency (SDe) and Neighbourhood Component Analysis with regularization (NCA-R) were compared. NCA-R was selected because its low cross-validation error resulted in significantly fewer features. NCA-R learn the weights of features by minimising an objective function which measures the average leave-one-out (LOO) classification or regression loss over the training data and fine-tuning a regularisation parameter ‘λ’ to 0.0329 while evaluating the weights of the features such that the significance is indicative of the rank. The features were selected using NCA-R for: (i) selecting subjects as ataxic or not (NCA_{RC}) (ii) regression with clinical scores as an estimation of the severity of ataxia (NCA_{RR}).

### Model Prediction

The most significant feature sets (NCA_{RC}, NCA_{RR}) selected by the NCA-R algorithm were then used in machine learning approaches to build models that could select (i.e. identify subjects as controls or individuals with FA) or compare against clinical scales (i.e. predict severity of ataxia according to clinical scales).

Model prediction for selection: A k-Nearest Neighbour (KNN) classification model was trained using (NCA_{RC}) features to classify subjects as individuals with FA or controls based on parameters such as accuracy and AUC from ROC.

Model prediction for comparison to clinical scores: A random-forest regression model (RFR) which uses fine-tree learning approach, was employed for enhancing prediction of clinical scores (mFARS) using NCA_{RR} features. Other models such as Random Subset regression, Multiple linear polynomial regression and the like were tested however using regression model parameters such as the goodness of fit measure R-squared (R²), mean absolute error (MAE) and correlation co-efficient P_{c}, the RFR performance was evaluated as the best performing. The classifier and regression model performance was validated using a 10 fold cross-validation technique in order to minimize misclassification and potential overfitting issues. Table 2 compares performance of the RFR based model prediction of mFARS with 10-fold cross validation (AIM-C) against 9HBT and BBT test.

TABLE 2

| | Predicted model parameters | | | Statistical parameters | | |
|---|---|---|---|---|---|---|
| Test | R² | MAE* | SDEr** | P_{c} | p-val | α(%) |
| AIM-C | 0.92 | 0.005 | 0.001 | 0.96 | <0.001 | 99.99 |
| 9HPT | 0.89 | 0.009 | 0.002 | 0.94 | <0.001 | 99.99 |
| BBT | 0.75 | 0.572 | 0.101 | 0.89 | <0.001 | 99.99 |

*Mean absolute error/loss after 10 fold cross-validation
**Standard deviation of mean absolute error

### Feature Categorisation

To categorise movement dysfunction, it is convenient to use known descriptions of the motor deficit of ataxia. Gordon Holmes's widely accepted descriptions can be conveniently categorised into four dimensions as follows: Stability (S), Timing (T), Accuracy (A) and Rhythmicity (R). In the present example, four propositions P1, P2, P3 and P4 described below were designed to enable each of the features identified in the movement data to be assigned one of the S, T, A and R dimensions. The primary axis is the vertical (X-axis) direction of motion. Resultant motion (X, Y, Z combined) of acceleration, and angular velocity were also considered as the primary motion and adheres to either proposition P1 or P4. Since uni-directional temporal variations of motion data were considered, principle P3 was not applicable for the pressure data.
P1: The direction of the movement execution for the task was considered the primary axis and any movement discrepancies in this axis contribute to the rhythmicity (R) dimension.
P2: The error/deviation from the most efficient path required to execute the task contribute to the accuracy (A) dimension.
P3: Dominant movements in axis other than the primary axis relate to secondary movements and contribute to the stability (S) dimension.
P4: Any errors in the primary axis in a temporal context, or delay in initiating the task contributes to the timing (T) dimension.

S.T.A.R. dimension assignment: Each selected feature was examined according to the above P1, P2, P3 and P4 propositions and assigned one of the dimensions S, T, A, or R. According to P1, the strength of the repetitiveness or magnitude at resonant frequency along the primary motion of the task movement were identified for the Rhythmicity dimension. According to P2, the features demonstrating deviations in the efficient path required to execute the task were identified for the Accuracy dimension. According to P3, features manifesting in deviations in directions other than the direction of dominant motion were identified as being in the Stability dimension. According to P4, the features which are associated with delay or timing deficit while carrying out the device accommodation or transport phase of the task were identified as being in the Timing dimension.

S.T.A.R contribution of the selected features: The S.T.A.R dimension assignment was applied to the feature subset obtained for both selection (NCA_{RC}) and severity estimation (NCA_{RR}). Thus, subsequent to the feature selection, features obtained from the NCA-R algorithm were assigned to one of the S.T.A.R. dimensions using propositions P1 to P4 and summed (according to the feature weightings from the relevant model) to determine the collective contribution to each dimension of the S.T.A.R classification.

### Discussion

A preliminary examination reveals that direct measures of movement such as acceleration (Acc), angular velocity (Gyr), pressure (Pr) as well as derived measures velocity (Vel) and Euler angle (Euler) can separate controls from individuals with FA. The mean values for acceleration, angular velocity, Euler angles and velocity were lower in individuals with FA than controls and mean pressure value was higher. The maximum pressure value was also higher when individuals with FA (−0.1 psi) performed the task than control participants (−0.05 psi) as shown in FIG. 5. This indicates that individuals with FA execute the task more slowly and compress the device more during the task.

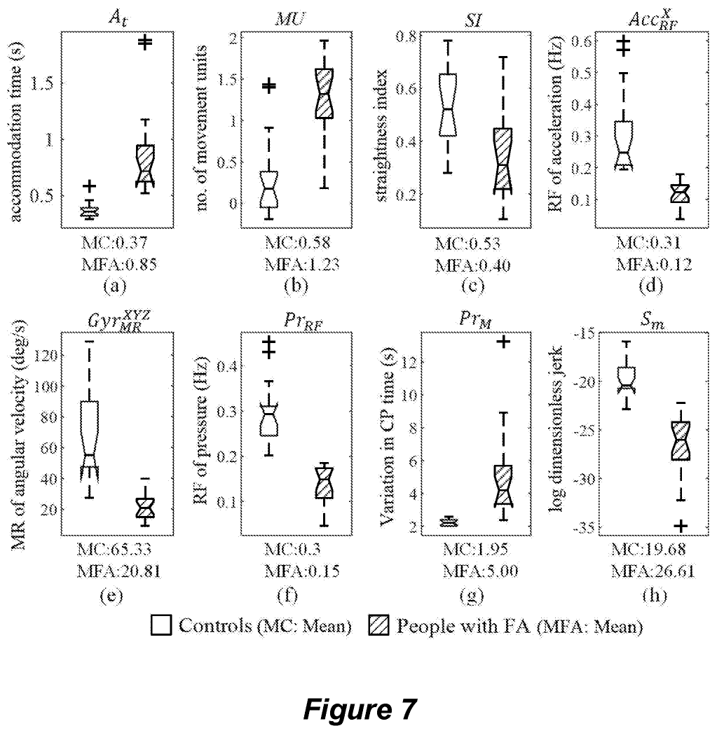

Diagnostic discrimination based on separation: Initially phase-of-movement based features were assessed as measures for the separating the two cohorts. FIG. 7 is a box plot representation of separation obtained using pressure and motion features. The features based on acquisition skills are shown in (a), (b), and (c). The frequency domain parameters of the IMU 210 (motion data) and pressure data are given in (d), (e), and (f). FIG. 7(g) depicts the time duration of compression and (h) is log dimensionless jerk.

The average accommodation time (AT) and the average number of movement units (MU) were greater when individuals with FA performed the task than when controls did (AT is 1 s and MU is 1.23 units respectively for individuals with FA compared to 0.3 s and 0.58 units for controls) (FIG. 7 (a,b)). A smaller number of MU (as measured in controls), implies straighter trajectory. Straightness index (SI) and initiation velocities were both higher in controls than individuals with FA (FIG. 7 (a,c)).

Frequency characteristics based features such as A_{CC}_{_{RF}}^{X} and Gyro_{MR}^{XYZ} (FIG. 7(d) and Table 3) acquired while individuals with FA were in the transporting phase are significantly distinct from those acquired while controls performed this phase of the task. These movements are performed at a lower frequency (f=0.12 Hz) by individuals with FA than by controls (f=0.3 Hz). The main differences were found along the X-axis (AUC≈1), which was the axis of primary motion during the execution of the task (Table 1). The cohort separation obtained from magnitude of angular velocity, Gyro_{MR}^{XYZ} (absolute angular velocity of X, Y and Z axes combined) may reflect efforts by ataxic subjects to make corrections in the line of motion and most likely reflect their instability while carrying the device to and from the mouth.

Smoothness (Sₘ) is considered to be a measure of the quality of motor performance, and is measured by dimensionless jerk. It was higher in movements made by controls (mean Sₘ=−19.7) than those made by people with ataxia (mean Sₘ=−26.6) from Table 3 and FIG. 7(h) and provided notable separation between the two cohorts (AUC≈1). The Euler angles also demonstrated significantly differentiated between movements of controls and individuals with FA, relating to irregularities in the orientation of the AIM-C device, particularly in θ and φ directions. This was particularly apparent in axis other than the main axis of motion and may have contributed to the incoordination observed by the clinician.

As noted above, individuals with FA compress the wall of the AIM-C device 200, more than controls (FIG. 5) and vary the pressure cycle more (AUC=0.80, Table 1). The differential pressure variation when described in the frequency domain (FIG. 7(g), Table 3) discriminated the two cohorts primarily based on the Pr_{RF} feature (with a mean of 0.3 Hz and 0.15 Hz for controls and subjects with FA respectively). This range of Pr_{RF} also confirmed that individuals with FA applied pressure to the flexible section of the canister for a longer duration (Table 3). Furthermore, the range of Pr_{M}, which is the variation of the mean duration of the compression phase, is greater for individuals with FA than it is for controls (FIG. 7 (g)). It can also be seen that, as the compression rate is increased, the task was executed at a relatively slower pace, i.e. as seen in FIG. 7(a,d,f,g), the mean of A_{CC}_{_{RF}}^{X} for controls and for individuals with FA are 0.3 Hz and 0.12 Hz respectively.

Individuals with FA could almost be completely distinguished from controls using Pr_{RF}, A_{CC}_{_{RF}}^{X}, Gyro_{MR}^{XYZ}, Sₘ, Aₜ and Pr_{M} features. Furthermore there was a marked difference in medians of these features in individuals with FA and controls, as evident in FIG. 7 (a,d,e,f,g,h) and Table 3. In contrast, features such as straightness index and number of movement units (FIG. 7 (b,c)) were of lesser diagnostic value.

Correlation assessment with clinical scores: Extracted pressure and motion features correlated significantly with clinical scores (Table 4). The individual feature with the highest correlation with mFARS was Pr_{RF} (S_{c}=0.87) and θ_{RF}^{c} (S_{c}=0.83). A notable finding of this study was that the features Pr_{SD}, Prᵣₘₛ, Pr_{MR}, Pr_{RF} correlated with the presence of spasticity (P_{c}=0.83) while maintaining significant cohort separation. It is hypothesised that these features may be important in

TABLE 3

| | Sₘ | | Pr_{RF} | | Acc_{RF}^{X} | | Gyr_{MR}^{XYZ} | | MU | | Aₜ | | SI | | Pr_{M} | |
|---|---|---|---|---|---|---|---|---|---|---|---|---|---|---|---|---|
| * | C | FA | C | FA | C | FA | C | FA | C | FA | C | FA | C | FA | C | FA |
| Min | −22.87 | −34.91 | 0.19 | 0.05 | 0.18 | 0.04 | 27.37 | 8.95 | 0.19 | 0.82 | 0.59 | 0.52 | 0.30 | 0.10 | 2.00 | 2.35 |
| Q1 | −20.69 | −28.05 | 0.25 | 0.11 | 0.22 | 0.1 | 48.05 | 14.94 | 0.56 | 0.58 | 0.33 | 0.64 | 0.52 | 0.32 | 2.01 | 3.42 |
| Median | 20.44 | −26.02 | 0.3 | 0.15 | 0.25 | 0.13 | 54.88 | 20.68 | 0.60 | 1.08 | 0.35 | 0.72 | 0.56 | 0.35 | 2.20 | 4.17 |
| Q3 | −18.76 | −24.3 | 0.31 | 0.18 | 0.35 | 0.15 | 89.11 | 26.26 | 0.80 | 1.69 | 0.38 | 0.94 | 0.68 | 0.49 | 2.39 | 5.62 |
| Max | −15.93 | −22.24 | 0.46 | 0.21 | 0.60 | 0.18 | 128.72 | 39.68 | 1.03 | 2.06 | 0.58 | 1.88 | 0.81 | 0.72 | 2.57 | 13.24 |
| Mean | −19.68 | −26.61 | 0.30 | 0.15 | 0.31 | 0.12 | 65.33 | 20.81 | 0.58 | 1.23 | 0.37 | 0.85 | 0.53 | 0.40 | 1.95 | 4.99 |
| Range | 6.94 | 12.68 | 0.26 | 0.16 | 0.41 | 0.15 | 101.36 | 30.73 | 0.84 | 1.98 | 0.29 | 1.36 | 0.51 | 0.19 | 0.57 | 1.08 |
| IQR | 1.94 | 3.76 | 0.07 | 0.07 | 0.14 | 0.05 | 41.06 | 11.33 | 0.23 | 1.11 | 0.06 | 0.31 | 0.16 | 0.17 | 0.37 | 2.20 |

* Descriptive statistics of the box-plot along with mean, range and Inter-Quartile Range (IQR)

TABLE 6

| | NCA_{RC} | | NCA_{RR} | | | | |
|---|---|---|---|---|---|---|---|
| Features | W | % Co* | W | % Co* | Pre** | Description | S.T.A.R |
| Pr_{RF} | 1.13 | 3.22 | — | — | P4 | timing deficit in the pressure compression rate during one complete cycle | T |
| Sₘ | 0.77 | 22.80 | — | — | P4 | error due to overshoots in the resultant path taken while executing the task | T |
| θ_{RF}^{c} | 0.53 | 15.72 | 0.43 | 23.35 | P3 | movement deficit due to uncoordinated secondary motion in θ direction | S |
| Aₜ | 0.32 | 9.47 | 0.52 | 28.01 | P4 | delay in moment initiation during grasping phase, to execute transportation phase | T |
| Gyr_{MR}^{XYZ} | 0.27 | 7.54 | — | — | P1 | movement deficit due to variation in magnitude of resultant rotational velocity | R |
| Acc_{RF}^{X} | 0.19 | 5.63 | — | — | P4 | timing deficit in the primary (X-axis) axis of movement execution | T |
| Pr_{M} | 0.12 | 3.46 | 0.90 | 48.64 | P4 | irregularities in the timing of compression phase | T |
| Ø_{RF}^{c} | 0.02 | 0.66 | — | — | 23 | movement deficit due to uncoordinated secondary motion in Ø axis | S |
| S_{T} | 0.02 | 0.63 | — | — | P2 | deviation from the end target during release phase to attain the stabilization phase | A |

S.T.A.R contribution for NCA_{RC} subset: Stability: 16.38%, Timing: 74.81%, Accuracy: 0.63%, Rhythmicity: 7.54%
S.T.A.R contribution for NCA_{RR} subset: Stability: 23.35%, Timing: 76.65%, Accuracy: 0.00%, Rhythmicity: 0.00%
‘W’ stands for feature weight,
‘—’ indicates features not selected in NCA_{RR} subset,
*Percentage feature contribution,
**Premisses of feature assignment

TABLE 4

| | mFARS total score/99 | | | 9HPT | | | ADL | | | Spasticity | | |
|---|---|---|---|---|---|---|---|---|---|---|---|---|
| | DC | PC | SC | DC | PC | SC | DC | PC | SC | DC | PC | SC |
| IMU data | | | | | | | | | | | | |
| Acc_{RF}^{X} | 0.7036 | 0.7631 | 0.8366 | 0.4387 | 0.4232 | 0.7983 | 0.6807 | 0.7573 | 0.8194 | 0.3245 | 0.3822 | 0.4296 |
| Acc_{MR}^{X} | 0.7190 | 0.6721 | 0.7165 | 0.4592 | 0.3582 | 0.7606 | 0.6890 | 0.6316 | 0.6623 | 0.3969 | 0.3256 | 0.3830 |
| Acc_{RF}^{Y} | 0.6686 | 0.8282 | 0.8421 | 0.2133 | 0.4846 | 0.8384 | 0.2768 | 0.8019 | 0.8269 | 0.2609 | 0.4891 | 0.4617 |
| Acc_{MR}^{X} | 0.4285 | 0.2450 | 0.1648 | 0.3521 | 0.1673 | 0.2883 | 0.4237 | 0.2157 | 0.1505 | 0.3425 | 0.1361 | 0.0174 |
| Acc_{RF}^{Z} | 0.6362 | 0.7950 | 0.8135 | 0.3631 | 0.4900 | 0.8034 | 0.4289 | 0.7799 | 0.7977 | 0.2916 | 0.4368 | 0.4454 |
| Acc_{MR}^{Z} | 0.6082 | 0.5617 | 0.5625 | 0.3752 | 0.2835 | 0.5787 | 0.6032 | 0.5501 | 0.5561 | 0.4993 | 0.3703 | 0.3830 |
| Acc_{RF}^{XYZ} | 0.8074 | 0.7581 | 0.8120 | 0.5128 | 0.4655 | 0.7715 | 0.7857 | 0.7411 | 0.8031 | 0.2946 | 0.3121 | 0.2937 |
| Acc_{MR}^{XYZ} | 0.8819 | 0.8426 | 0.8134 | 0.5093 | 0.4209 | 0.7926 | 0.8592 | 0.8101 | 0.7803 | 0.4008 | 0.3312 | 0.4004 |
| Gyr_{RF}^{XYZ} | 0.7116 | 0.6887 | 0.6432 | 0.4019 | 0.3359 | 0.6900 | 0.7093 | 0.6908 | 0.7011 | 0.2373 | 0.1603 | 0.2094 |
| Gyr_{MR}^{XYZ} | 0.8510 | 0.7619 | 0.8249 | 0.4984 | 0.3908 | 0.8682 | 0.8261 | 0.7343 | 0.7984 | 0.4349 | 0.4097 | 0.4178 |
| Ø_{RF}^{m} | 0.5953 | 0.8290 | 0.8303 | 0.1959 | 0.4612 | 0.8178 | 0.2882 | 0.8009 | 0.7995 | 0.1683 | 0.3841 | 0.4005 |
| Ø_{MR}^{m} | 0.2509 | 0.1850 | 0.1243 | 0.2595 | 0.0495 | 0.1700 | 0.2427 | 0.1500 | 0.0929 | 0.2197 | 0.0745 | 0.1044 |
| θ_{RF}^{m} | 0.5730 | 0.6519 | 0.6267 | 0.1827 | 0.3523 | 0.6706 | 0.1840 | 0.6121 | 0.5783 | 0.2840 | 0.4067 | 0.4132 |
| θ_{MR}^{m} | 0.3942 | 0.3491 | 0.4029 | 0.2641 | 0.1925 | 0.3694 | 0.3577 | 0.2963 | 0.3104 | 0.2765 | 0.1745 | 0.2785 |
| ψ_{RF}^{m} | 0.4763 | 0.6592 | 0.7840 | 0.3006 | 0.3072 | 0.7669 | 0.4572 | 0.6405 | 0.7364 | 0.1852 | 0.3332 | 0.4120 |
| ψ_{MR}^{m} | 0.4257 | 0.2206 | 0.2337 | 0.3353 | 0.1991 | 0.1390 | 0.4261 | 0.1724 | 0.1567 | 0.3910 | 0.3462 | 0.4178 |
| Ø_{RF}^{c} | 0.6770 | 0.5877 | 0.8441 | 0.1578 | 0.3298 | 0.8360 | 0.1714 | 0.5696 | 0.8280 | 0.4288 | 0.5432 | 0.5063 |
| Ø_{MR}^{c} | 0.4317 | 0.3918 | 0.3878 | 0.3021 | 0.2383 | 0.3586 | 0.4428 | 0.3961 | 0.3608 | 0.2665 | 0.1942 | 0.2263 |
| θ_{RF}^{c} | 0.6659 | 0.8345 | 0.8348 | 0.3930 | 0.4752 | 0.8012 | 0.6439 | 0.8303 | 0.8176 | 0.2006 | 0.3822 | 0.4296 |
| θ_{MR}^{c} | 0.5123 | 0.5045 | 0.5369 | 0.3665 | 0.3034 | 0.5337 | 0.4837 | 0.4603 | 0.4614 | 0.3185 | 0.2990 | 0.2959 |
| Ø_{RF}^{k} | 0.3060 | 0.8493 | 0.8124 | 0.2346 | 0.4337 | 0.7879 | 0.3099 | 0.8183 | 0.7939 | 0.4685 | 0.4646 | 0.4529 |
| Ø_{MR}^{k} | 0.2578 | 0.1162 | 0.4049 | 0.2828 | 0.2048 | 0.3717 | 0.2870 | 0.1772 | 0.4491 | 0.1889 | 0.1181 | 0.0870 |
| θ_{RF}^{k} | 0.6086 | 0.8345 | 0.8348 | 0.3705 | 0.4752 | 0.8012 | 0.5881 | 0.8303 | 0.8176 | 0.2533 | 0.3823 | 0.4296 |
| θ_{MR}^{k} | 0.5245 | 0.5117 | 0.5343 | 0.3594 | 0.2851 | 0.5401 | 0.4960 | 0.4681 | 0.4587 | 0.3359 | 0.2832 | 0.2611 |
| Sₘ | 0.8412 | 0.8041 | 0.8099 | 0.4986 | 0.4129 | 0.7879 | 0.8206 | 0.7938 | 0.7971 | 0.2584 | 0.1540 | 0.1219 |
| Pressure data | | | | | | | | | | | | |
| Prₘ | 0.2069 | 0.0782 | 0.0006 | 0.2045 | 0.0654 | 0.0621 | 0.2220 | 0.0317 | 0.0234 | 0.5629 | 0.5956 | 0.5378 |
| Prᵣₘₛ | 0.2131 | 0.1515 | 0.2126 | 0.3300 | 0.2510 | 0.2381 | 0.2654 | 0.1858 | 0.2383 | 0.7681 | 0.7470 | 0.7360 |
| Rr_{RF} | 0.8913 | 0.8634 | 0.8755 | 0.5566 | 0.4749 | 0.8289 | 0.8697 | 0.8141 | 0.8359 | 0.6919 | 0.8376 | 0.7359 |
| Pr_{MR} | 0.8224 | 0.8008 | 0.8111 | 0.6773 | 0.5283 | 0.7458 | 0.7764 | 0.7176 | 0.7459 | 0.5975 | 0.8026 | 0.7223 |
| Pr_{BW} | 0.7770 | 0.7229 | 0.6968 | 0.5096 | 0.4633 | 0.7809 | 0.7679 | 0.6963 | 0.6611 | 0.3828 | 0.4501 | 0.3962 |
| Pr_{SD} | 0.6607 | 0.6175 | 0.7397 | 0.5640 | 0.4772 | 0.7370 | 0.6942 | 0.6436 | 0.7762 | 0.6782 | 0.7865 | 0.5944 |
| Pr_{CP/UP} | 0.2222 | 0.1516 | 0.2382 | 0.3700 | 0.3511 | 0.1846 | 0.2145 | 0.1319 | 0.1834 | 0.3329 | 0.3237 | 0.2832 |
| Pr_{M} | 0.8584 | 0.7741 | 0.8568 | 0.5565 | 0.5241 | 0.8326 | 0.8666 | 0.7820 | 0.8610 | 0.3822 | 0.3936 | 0.3963 |
| Combined | | | | | | | | | | | | |
| NCA_{RR} | 0.9666 | 0.9676 | 0.9032 | 0.7321 | 0.7158 | 0.8491 | 0.8225 | 0.7973 | 0.8303 | 0.4998 | 0.4609 | 0.4661 |
| SFS^{c} | 0.8509 | 0.8643 | 0.8326 | 0.4951 | 0.3960 | 0.8278 | 0.8105 | 0.7403 | 0.8099 | 0.4349 | 0.4449 | 0.4908 |
| RSFS^{c} | 0.8708 | 0.8642 | 0.8326 | 0.3957 | 0.4948 | 0.8278 | 0.8105 | 0.7402 | 0.8099 | 0.4346 | 0.4751 | 0.4067 |
| SDe^{c} | 0.8368 | 0.8622 | 0.8322 | 0.3949 | 0.4940 | 0.8578 | 0.8343 | 0.7402 | 0.8010 | 0.4341 | 0.4763 | 0.4067 |
| PR^{c} | 0.8820 | 0.8857 | 0.8325 | 0.7216 | 0.7056 | 0.8211 | 0.8198 | 0.7946 | 0.8335 | 0.4998 | 0.4609 | 0.4661 |
| AccGr^{c} | 0.8911 | 0.8890 | 0.8420 | 0.5175 | 0.7158 | 0.4506 | 0.8086 | 0.8649 | 0.8303 | 0.2747 | 4524 | 4343 |

DC: Distance correlation,
PC: Pearson correlation,
SC: Spearman rank correlation

describing the inability in ‘accommodating’ the AIM-C device 200 in the hand during the movement task.

Features A_{CC}_{_{RF}}^{XYZ} (S_{c}=0.81), Gyro_{MR}^{XYZ} (S^{c}=0.80) and Pr_{RF} (S^{c}=0.84) correlated highly with clinical scales of ADL (Table 4). This implies that both pressure and motion features extracted using the AIM-C device 200 measure movement impairments that interfere with tasks performed in everyday life. It is notable in Table 4 that a correlation with the 9HPT was only obtained with the Spearman rank correlation technique, which may reflect non linearity in the 9HPT measurements.

Since most features separated individuals with FA from controls and provided significant p-values and high correlations (Table 4), the NCA-R feature selection scheme with 10-fold cross validation was used to find the best features in terms of diagnostic and severity prediction as discussed below.

Feature Selection for Classification and Severity Estimation Using NCA-R with Predictive Modelling

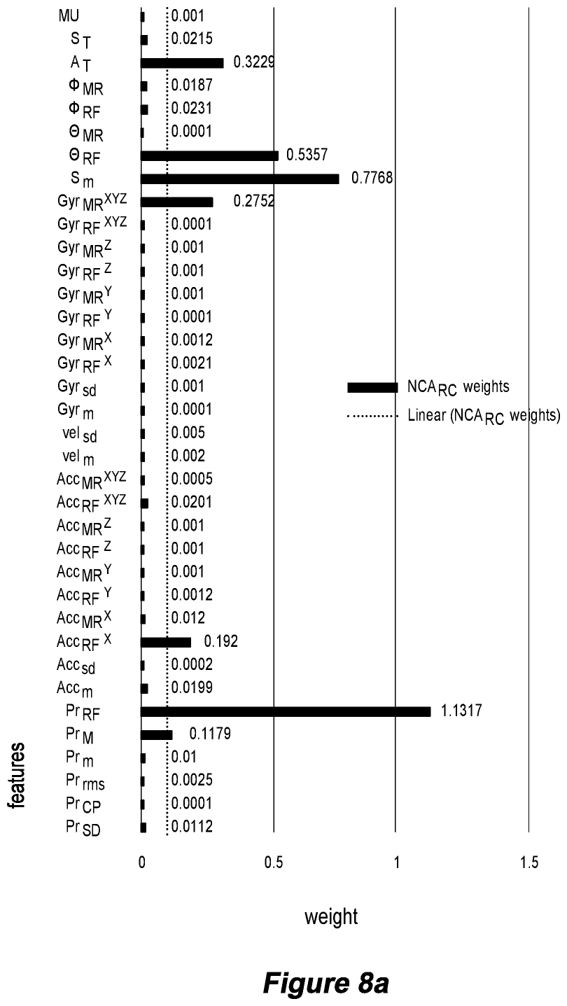

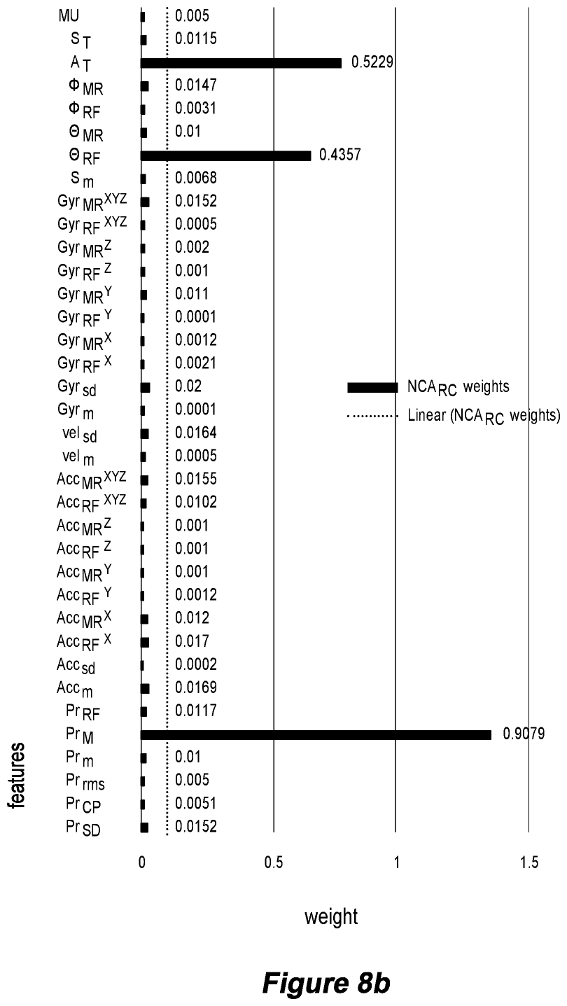

FIG. 8 is a chart showing weights obtained for (a) NCA_{RC} subset (b) NCA_{RR} subset using NCA-R selection. During the NCA-R feature selection process, only the features that contributed more than 2% of the maximum feature weight were selected for diagnosis and for severity assessment. The feature subsets (NCA_{RC}) selected for classification include Pr_{RF}, θ_{RF}^{c}, A_{CC}_{_{RF}}^{X}, Sₘ, Aₜ, S_{T}, Pr_{M}, Ø_{RF}^{c}, Gyr_{MR}^{XYZ}, (FIG. 8). Based on the features in NCA_{RC} subset the inventors have found that the AIM-C device 200 captured information about different movement deficits that discriminated individuals with FA from controls.

Using NCA_{RC} subset trained with a KNN classifier, the information and data obtained from the AIM-C device 200 correctly classified control participants (specificity≈1) from individuals with FA (sensitivity≈1) with high diagnostic accuracy (99%). Table 5 shows a comparison of KNN with other classification models with NCA_{RC} subset after 10 fold cross-validation, where SVM is Support Vector Machine classifier, RFC is Ensemble Tree classifier, LDA is Linear discriminant classifier. ACC is classification accuracy, TPR (sensitivity) represents: the proportion of individuals with FA identified as having the condition, TNR (specificity) represents the number of controls correctly classified as having no condition, PPV: represents precision, No. represents number of features.

TABLE 5

| Selection | No. | ACC % | AUC | TPR | TNR | PPV |
|---|---|---|---|---|---|---|
| KNN | 9 | 99 | 0.99 | 0.99 | 0.99 | 0.99 |
| SVM | | 93 | 0.98 | 0.96 | 0.98 | 0.96 |
| RFC | | 98 | 0.98 | 0.98 | 0.99 | 0.98 |
| LDA | | 88 | 0.96 | 0.90 | 0.92 | 0.90 |

The feature subset (NCA_{RR}) selected for severity assessment included Pr_{M}, Aₜ and θ_{RF}^{c}. The subset NCA_{RR} when trained with RFR, predicted mFARS (Table 2) with a high goodness of fit parameter (R²=0.92) and superior correlation (P_{c}=0.96) with mFARS scores. A severity prediction model of 9HPT and BBT provided a lower R² and correlation value (Table 2).

The power of NCA_{RC} and NCA_{RR} feature sets were 99.99% with an effect size of 1.182 (Table 1) which was greater than the required statistical power of 80% for the sample size.

### S.T.A.R Assessment:

Each feature in either the NCA_{RC} or NCA_{RR} subset can be allocated to one of the four propositions P1, P2, P3 and P4. The feature-weights from the relevant model (KNN or RFR) can then be used to determine the percentage contribution of each feature to the dimensions of the S.T.A.R. descriptions which in turn provide clinical context for describing the manifestation of the movement disorder. Table 6 shows the S.T.A.R dimension assignment for the NCA_{RC} and NCA_{RR} feature subsets selected using NCA-R algorithm.

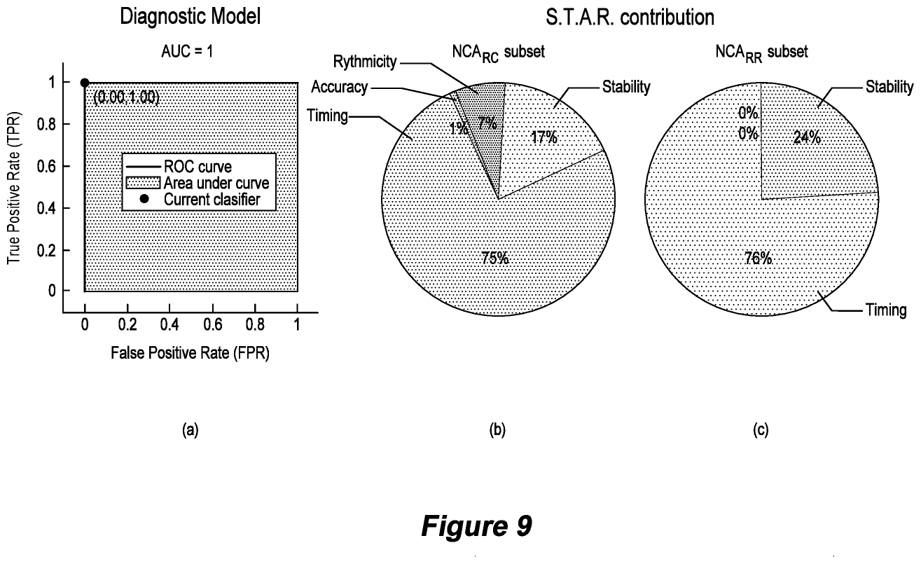

For the NCA_{RC} subset, the Timing (T) dimension contributed the most (75%) (Table 6 and FIG. 9(b)). Features pertaining to the Stability (S) dimension contributed 17% to accurately identifying a person with FA. The Rhythmicity (R) dimension contributed 7% while the Accuracy (A) dimension contributed the least (1%).

For the NCA_{RR} subset, the Timing (T) dimension contributed the most again (76%) (Table 6 and FIG. 9(c)). Features pertaining to the Stability (S) dimension had the next highest contribution (24%) in estimating the severity of a person with FA. The Rhythmicity (R) and Accuracy (A) dimensions failed to contribute substantially to the S.T.A.R dimensions for the NCA_{RR} subset.

The S.T.A.R. assessment approach has provided objective indications that domains such as timing and stability provide significant contributions to both diagnostic accuracy and severity estimation, and point to differences between ataxic and non-ataxic movement when maintaining the stability of the execution platform.

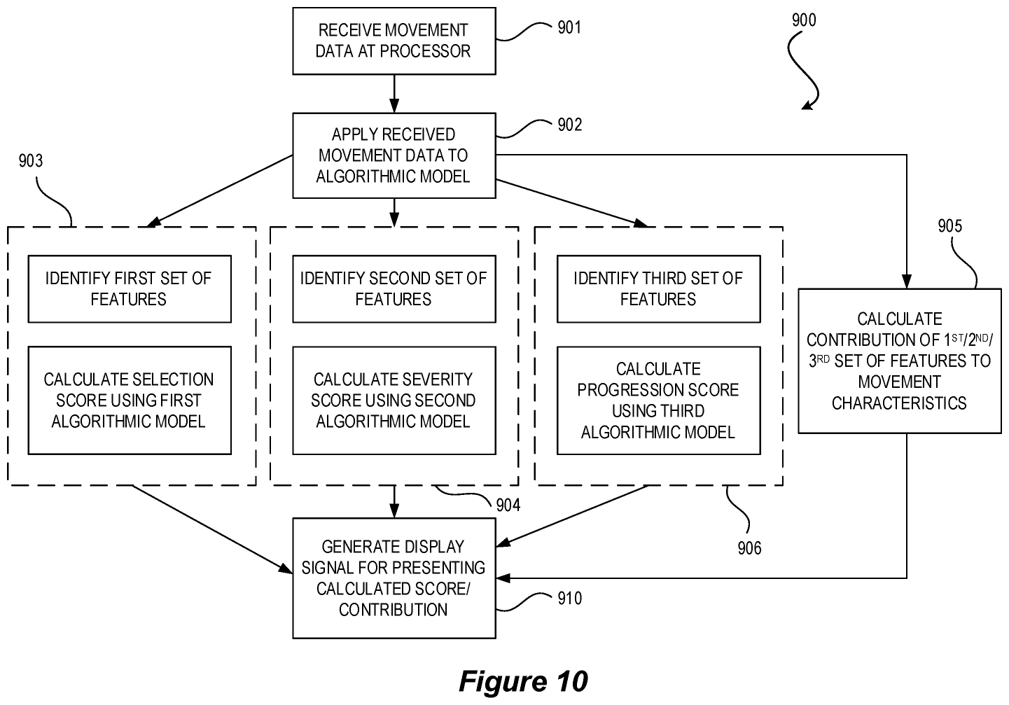

Thus, embodiments of the disclosure provide an automated method as shown in the schematic illustration of FIG. 10 for objectively quantifying a motor control disorder in a subject. The method comprises, in a step 901 receiving at a processor movement data corresponding to movements of a limb of the subject and, in a step 902, the processor applying the received movement data to an algorithmic model and identifying one or more features from the movement data that represent disordered movement in the subject. The processor then calculates from the one or more identified features, a score quantifying the motor control disorder in the subject. The calculated selection and/or severity scores may be scaled to correspond to an accepted clinical scale such as FARS, mFARS, 9HPT, BBT, SARA, ICARS, ADL or the like, or to some other clinically meaningful reference.

In preferred embodiments, the received movement data is collected while the subject performs a movement task which is or simulates an activity of daily living such as eating or drinking. The movement data comprises pressure data and motion data obtained from a movement detection device. Typically, the movement detection device comprises a pressure sensor for monitoring pressure applied to the device during a movement task, and motion sensors such as an accelerometer and gyroscope monitoring kinematic motion of the device in multiple axes. In one embodiment, the motion sensors are provided in the form of an IMU.

In a step 903, the processor applies the received movement data to a first algorithmic model to identify a first set of features used by the processor to calculate a selection score which is indicative of presence or absence of the motor control disorder in the subject. Where the motor control disorder is Ataxia, the first set of features may comprise a single feature, ideally a pressure feature such as Pr_{RF}. In other embodiments, the first set of features comprises some or all of the features Pr_{RF}, A_{CC}_{_{RF}}^{X}, Gyro_{MR}^{XYZ}, Sₘ, Aₜ and Pr_{M}. In a preferred embodiment, the feature set comprising θ_{RF}^{c}, A_{CC}_{_{RF}}^{X}, Sₘ, Pr_{RF}, Aₜ, S_{T}, Pr_{M}, Ø_{RF}^{c} and Gyr_{MR}^{XYZ} provides a selection score yielding high identification of FA (99%). Such features are described in Table 1.

In another step 904, the processor applies the received movement data to a second algorithmic model to identify a second set of features used by the processor to calculate a severity score which is indicative of severity of the motor control disorder in the subject. Where the motor control disorder is Ataxia, the second set of features may comprise a single feature, or a plurality of or all of the features selected from the group comprising: Pr_{RF} Pr_{M}, Aₜ and θ_{RF}^{c}. In one embodiment, the feature set comprising Pr_{M}, Aₜ and θ_{RF}^{c} is preferred.

In a step 910 the processor generates a display signal causing the calculated selection and/or severity scores to be presented on a display device such as a screen of a computer, smart phone, tablet or the like, or presented in a report transmitted to an operator by email or other suitable communication means.

In a step 905, the processor categorises movement dysfunction in the subject, by calculating a contribution made by each of the first, second or third (see Example 2) set of features to each of a plurality of movement characteristics that are attributable to movement dysfunction in the subject. In one embodiment, the plurality of movement characteristics are determined according to propositions P1 to P4 disclosed herein and the contribution made by the features to each of the propositions may be aggregated and presented on the display device. In another embodiment, propositions P1 to P4 may mapped to clinically accepted descriptions or categories of movement disorder which, in the case of Ataxia, can be conveniently categorised into four dimensions as follows: Stability (S), Timing (T), Accuracy (A) and Rhythmicity (R). Thus, the contribution of each of the S, T, A and R dimensions in the movement data may be presented in a step 910 on the display device e.g. as percentages and/or rankings or other measures that represent the dominance of each dimension on the movement.

While the example herein demonstrates utility in objectively detecting the presence of and scoring the severity of ataxia, it is to be understood that the embodiments disclosed also have utility in assessing other aspects of motor control disorder such as spasticity. In some embodiments, one or more or all of the features Pr_{SD}, Pr_{RMS}, Pr_{MR}, Pr_{RF} as described in Table 1 can be used to identify the presence/absence of spasticity.

### Example 2—Progression Score

Ten individuals diagnosed with FRDA (mean standard deviation (SD) age=37.05±12.23, 4 males and 6 females) were assessed at two time points (T1 and T2) that were on average 24 weeks apart. At T1, the average Functional Staging of Ataxia score of participants was 4.8 (SD 0.42), indicating moderate disability (walking requires an assistive device) and 68.3 (SD 10.01) on the mFARS. Table 7 shows clinical features of the cohort at T1 and T2 (GC: Group characteristics, M: mean, SD: standard deviation, R: Range, n: number of participants (n=10), age is given in years, no. of males: 4, females: 6; right hand dominant: 10, * is age of onset in years, + is disease duration in years).

TABLE 7

| | T1 | | | T2 | | |
|---|---|---|---|---|---|---|
| GC | M | SD | R | M | SD | R |
| Age | 38.5 | 13 | 19-60 | 40.1 | 15.15 | 19-60 |
| Onset* | 16.3 | 7.6 | 6-34 | — | — | — |
| DDu⁺ | 19.5 | 10.5 | 2-39 | — | — | — |
| GAA1 | 732.5 | 253.5 | 383-1298 | — | — | — |
| GAA2 | 917.7 | 235.1 | 611-1298 | — | — | — |
| Staging | 4.80 | 0.42 | 4-5 | 4.94 | 0.39 | 4-5.5 |
| mFARS | 68.3 | 10.1 | 47-85 | 68.2 | 8.7 | 46-82 |
| ADL | 19.8 | 3.4 | 16-25 | 21.1 | 3.4 | 15.5-24.5 |
| 9HPT | 69.9 | 30.1 | 30-119 | 83.7 | 29.8 | 52-149 |
| BBT | 20.6 | 6.8 | 4-31 | 21.3 | 6.9 | 7-29 |

Additionally, all participants performed a task that simulated drinking from the AIM-C device 200 while movement data was recorded by sensors in the canister as described previously. Recordings were made at T1 (first visit) and T2 (second visit) approximately 24 weeks later. At each visit, clinical assessments including Functional Staging of Ataxia score (Staging), mFARS, 9HPT and BBT were administered specifically. The upper limb subscale from the mFARS (NeuroUL) was used in the analysis. Other clinical parameters were collected, included GAA1 repeat size (GAA repeat size on the smallest allele) and GAA2 repeat size (GAA repeat size on the largest allele), age of disease onset (the age that symptoms related to FRDA were first observed), and disease duration (difference between current age and age of disease onset).

Using Wilcoxin-signed rank statistical test (with 1−αₛ=0.01) on a sample size of 10 subjects, the effect-size (d) of 1.59 was obtained after feature selection with a power P_{α}≥80%. Hence, the sample size of 10 was considered adequate for this study.

### Signal Pre-Processing

The kinematic measures obtained from the IMU were acceleration (acc) and angular velocity (gyr) while the kinetic measure obtained from the pressure sensor was the differential pressure (pr) representing the grip pressure exerted on the AIM-C device 200. Both measures were sampled at 100 Hz and low pass filtered with a cut-off frequency of 20 Hz. A median filter was used to suppress noise transients in the data. A 6th order Savitzky-Golay filter smoothed data and contributed to minimising the drift effects with averaging.

### Feature Extraction

Spatio-temporal features of kinematic measurements were obtained using a complimentary filtering technique based on Madgwick's algorithm. This technique extracted Euler angles (roll φ), pitch (θ) and yaw (Φ) as ‘deduced’ measures with the gravitational contributions removed while converting the sensor frame measurements to the global frame. The velocity of movement was a ‘deduced’ measure obtained from acceleration data after the bias removal.

The features were considered in the time, frequency, and time-frequency domains. Twenty eight features of interest were extracted using the following described signal processing techniques to obtain time domain features, frequency domain features, and time-frequency domain features.

A) Time Domain Features: Time domain features from the kinematic and kinetic data included statistical parameters such as mean and standard deviation. Range of Motion (ROM) of the AIM-C device 200 as it was transported from the table to the lip was obtained as the difference between highest and lowest position of the pitch (elevation) angle for the time duration T_{N} over five cycles.

$${ROM}_{θ} = {max}_{t ∈ [ 1 , {T}_{N} ]} {θ}_{t} - {min}_{t ∈ [ 1 , {T}_{N} ]} {θ}_{t} \qquad ( 6 )$$

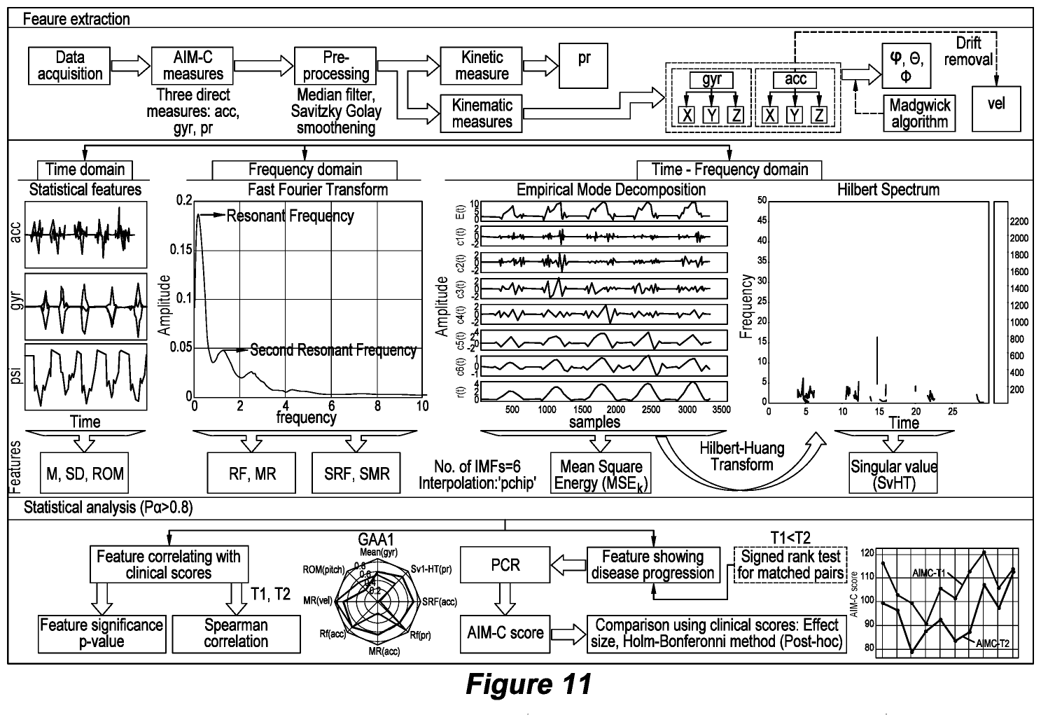

B) Frequency Domain Features: In the frequency domain analysis, the spectral leakages were reduced using Blackman-Harris windowing technique to improve the algorithmic performance when capturing features such as resonant frequency (RF) and magnitude at resonance (MR). The frequency domain features such as RF and MR were extracted for both kinematic (in all three orthogonal X, Y and Z axes combined) and kinetic measures. FIG. 11 is a schematic illustration exemplifying an approach to feature extraction for obtaining a progression score (represented as AIMC-T1 and AIMC-T2). The RF and MR features were also extracted for deduced measures such as velocity and (p, e, CD angles. After smoothing the kinetic and kinematic measures, the second highest peak (referred to as ‘Second Dominant Peak’ (SDP)) other than the MR in the frequency domain was used to determine secondary motion or oscillations. The second dominant peak analysis extracted features in the forms of Second Resonant Frequency (SRF) and Second Magnitude at Resonance (SMR). Also, the Mean value of Second Resonant Frequency (MSRF) was calculated for the cohort at T1 and T2.

C) Time-frequency domain features: Only the linear and periodic behaviour of the data was extracted by frequency domain analysis. However it is possible that disease progression is related to nonlinear and non-stationary behaviour of the kinetic and kinematic measures. Hence, the Hilbert-Huang Transform (HHT) which characterises non-linear behaviour by ‘time-frequency’ domain variations was used to probe the movement data.

The HHT combines two methods: First, an Empirical Mode Decomposition (EMD) to decompose the time series into Intrinsic Mode Functions (IMF). EMD consisted of a sifting process of the original signal, in which ‘n’ IMFs were obtained for each of the measures. Second, the Hilbert transform was used to compute instantaneous amplitude and frequency and present the IMF's in an energy-time-frequency domain representation. The application of HHT algorithm on EMD as described by Huang et al. is given as follows.

Consider n=6 IMFs (Cₖ(t), . . . , Cₙ(t)∀k∈[1, 2, . . . n]) and a residue signal rₙ(t). The decomposed signal of each of the measures (acc, gyr, pr) denoted by E(t) is given by:

$${E}_{t} ( t ) = {\sum }_{k = 1}^{n} {C}_{k} ( t ) + {r}_{n} ( t ) . \qquad ( 7 )$$

The Hilbert transform Hₖ(t) of each IMF Component Cₖ(t) is:

$${H}_{k} ( t ) = \frac{1}{π} {\int }_{- ∞}^{+ ∞} \frac{{C}_{k} ( τ )}{t - τ} d τ . \qquad ( 8 )$$

The analytic signal is then defined as:

Rₖ(t)=Cₖ(t)+jHₖ(t)=αₖ(t)e^{jØ(t)} (9)

where the amplitude αₖ(t)=\sqrt{Cₖ(t)²+Hₖ(t)²} and the phase

$$ϕ ( t ) = arctan [ \frac{{H}_{k} ( t )}{{C}_{k} ( t )} ] .$$

Corresponding to this, the instantaneous frequency can be given as:

$${ω}_{k} ( t ) = \frac{d {ϕ}_{k} ( t )}{d t} . \qquad ( 10 )$$

The Hilbert spectrum representing amplitude on the time-frequency domain is given by:

H(ω,t)=Re[Σₖ₌₁ⁿαₖ(t)e^{j∫ω}^{ₖ}^{(t)dt}] (1)

Features extracted in time-frequency domain. The Mean Square Energy (MSE) of the k^{th} IMF is given by,

$${MSE}_{k} = {\sum }_{𝔱 = 0}^{T} \frac{{C}_{k} ( t )}{N} , \qquad ( 12 )$$

where T is the sampling time, N is the length of the series and k is the IMF index. MSEₖ∀k∈[1, 2, . . . , 6] is considered to be a feature that might represent changes in disease progression.

A z×q matrix was obtained as the HHT spectrum, where z corresponds to frequency sampling points, and q the time sampling points. In combination with HHT, a Singular Value Decomposition (SVD) technique was further engaged for extracting features (this approach is hereinafter referred to as SvHT). The singular values of HHT spectrum were extracted using SVD, where SVD decomposes the matrix into three: one diagonal matrix and two orthogonal matrices. The SVD of the z×q matrix A is given as:

A=U\sqrt{λ}V^{T}, (13)

where U and V are orthogonal matrices of size z×z and q×q respectively, and \sqrt{λ} is a diagonal matrix of singular values. The first ten singular values i.e. \sqrt{λ}=[Sᵥ₁, Sᵥ₂, Sᵥ₃ . . . Sᵥ₁₀]T of the HHT matrix were extracted as features where the initial values contained most of the matrix information and can be related to the energy of the measure. For clarity, notational abbreviations of extracted features using the techniques described (FFT, EMD, HHT) are given in Table 8, noting the three direct measures utilised in this Example were acc, gyr and pr.

TABLE 8

| Method | Notation | Feature Description |
|---|---|---|
| Time | measureₘ | Mean value of the measure |
| | measure_{sd} | Standard deviation of the measure |
| FFT | MRₘₑₐₛᵤᵣₑ | Magnitude at resonance of the measure |
| | RFₘₑₐₛᵤᵣₑ | Resonant frequency of the measure |
| | SMRₘₑₐₛᵤᵣₑ | Second magnitude at resonance of measure |
| | SRFₘₑₐₛᵤᵣₑ | Second resonant frequency of the measure |
| EMD | IMFᵢ^{measure} | iₜₕ intrinsic mode function of the measure |
| | | ∀i ∈ [1, 2, . . . n]) |
| | MSR_{IMF}_{ᵢ}^{measure} | Mean square energy of the iₜₕ |
| | | IMF of the measure |
| HHT | Sᵥ_{_{q}}-HTₘₑₐₛᵤᵣₑ | Singular value of HHT of the measure |
| | | ∀q ∈ [1, 2, . . . 20] |

### Statistical Analysis and Feature Selection

A) Feature selection and combination for AIM-C score for progression. The most important features contributing to disease progression were identified based on two premises. Firstly, the feature indicating change in value at T2 is given by

$${F}_{i}^{PC} = ( \frac{{F}_{i}^{T 2}}{{F}_{i}^{T 1}} - 1 ) , \qquad ( 14 )$$

where Fᵢ^{T1}, Fᵢ^{T2} are feature values at T1 and T2 respectively ∀i∈[1, 2, . . . Q] (Q is the number of features extracted). Secondly, the statistical significance of the change from T1 to T2 is assessed by the Wilcoxon signed-rank test for matched pairs since not all data sets were normally distributed. The Wilcoxon signed rank test found the difference between each set of matched pairs (T1 and T2) in terms of error rate as and power P_{α}.

The features adhering to these two premises were selected as the best features. Amalgamation of these features was used to generate a score that represents the overall disease progression. This was achieved through Principal Component Analysis (PCA) followed by linear regression, collectively denoted as Principal Component Regression [43]. The dimensionality reduction of all combined features after normalisation was accomplished through the Principal Component Analysis (PCA), using visual observation of data distributions. Using linear orthogonal transformation, the data is transformed into a new coordinate system where the diagonal covariance matrix maximizes feature variance and then projects the data in accordance with the variance of the distributions. The Principal Component Regression algorithm consisted of passing the first principal component (representing the maximum variance of the feature contribution) to a linear regression to generate the AIM-C score for progression.

B) Comparison of AIM-C score for progression and clinical parameters used to measure performance overtime (mFARS, BBT 9HPT). The sensitivity of AIM-C score for progression to detect changes in scores at T2 compared to T1 was statistically quantified using the effect-size (d) (using G*Power software). The capacity of data from the AIM-C device 200 to capture change over time was compared to the mFARS, BBT and 9HPT. The differences between multiple groups (mFARS, BBT, 9HPT and AIM-C scores) were compared using the Holm-Bonferroni multi comparison post-hoc method to compare the (T1 and T2) scores that exhibited significant difference to each other. This method was also robust against type-I errors by controlling the family-wise error rate (FWER) at 0.05 for Holm-correction.

C) Feature correlation with clinical parameters. All the kinetic and kinematic features extracted were also correlated with the clinical measures (mFARS score, the NeuroUL score, the Functional Staging of Ataxia score, ADL score, 9HPT and BBT) and clinical parameters (GAA1, GAA2, disease duration and age at disease onset) at T1 and T2 using Spearman's rank correlation (P_{c}^{T1 or T2}). The functional architecture of the overall methodology for Example 2 is provided in FIG. 11.

Features Extracted to Generate AIM-C Score and Comparison with Clinical Scores

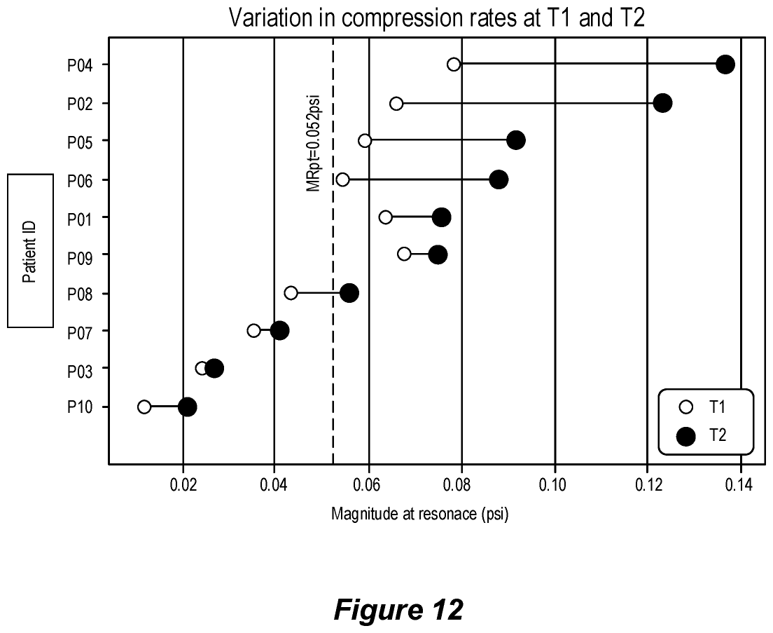

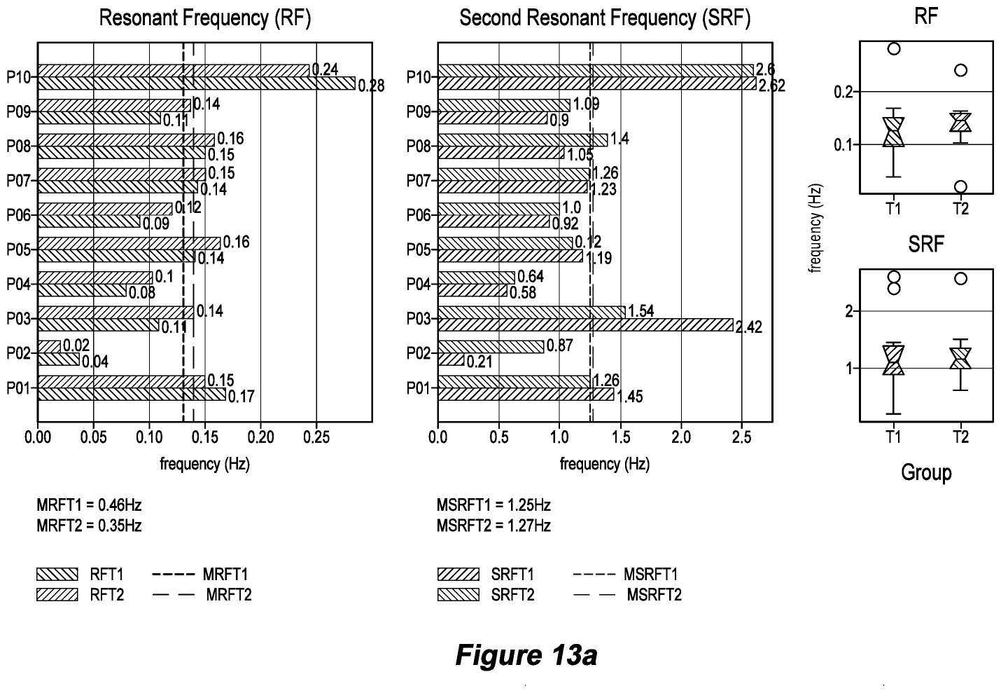

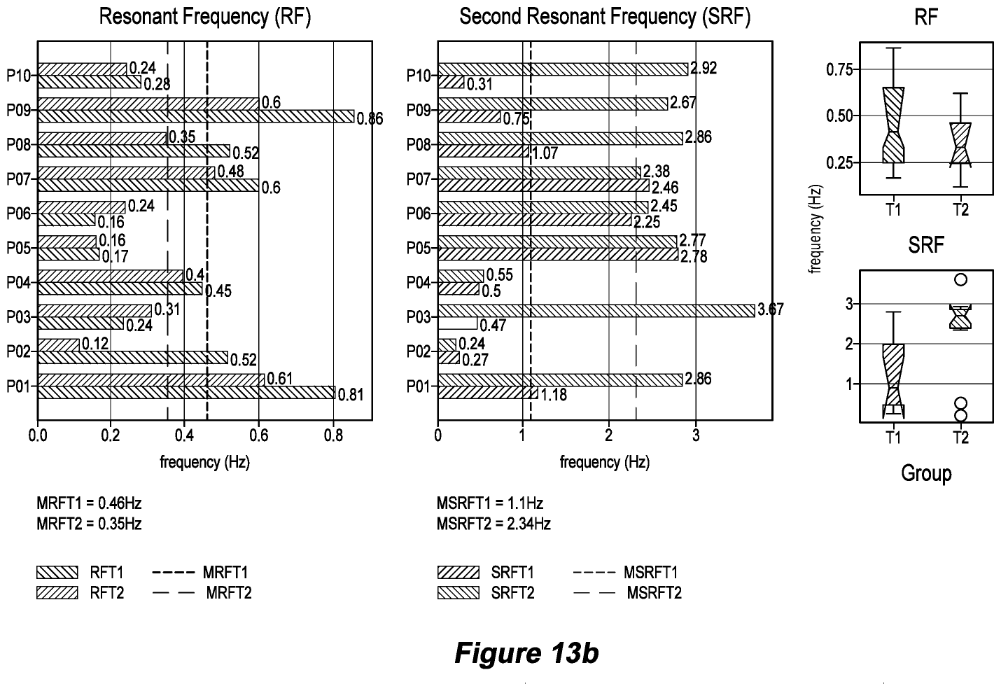

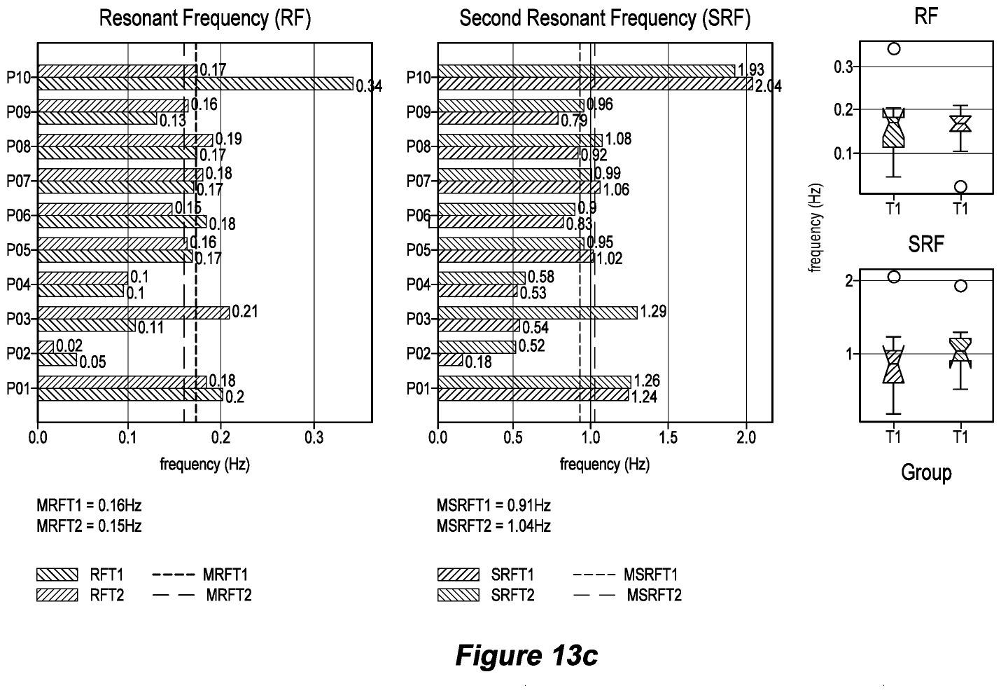

A) Extracted features: The MRₚᵣ feature value obtained from the frequency analysis of all participants increased from timepoint T1 to T2. This is shown in FIG. 12 which shows the magnitude at resonance for MRₚᵣ showing a change in compression rate. The frequencies of the second highest peaks (SRF) of the kinetic and kinematic data at T1 and T2 was compared as shown in FIG. 13 which shows the difference between T1 and T2 values of RF and SRF of (a) acceleration, (b) angular velocity, and (c) pressure. The mean SRF value of all three measures were higher at T2 than at T1. The change in SRF from T1 to T2 differs from the respective change in RF from T1 to T2. The increase in the frequency from T1 to T2 of the feature SRF_{gyr} was the highest and was notably different compared to RF_{gyr}. This is evident in Table 9 which shows the range of measured resonant frequencies at the first (RF) and the second dominant peaks (SFR), as well as in FIG. 13.

TABLE 9

| Frequency | Range of:acc | Range of:gyr | Range of:pr |
|---|---|---|---|
| RF* (Hz) | 0.01-0.18 | 0.10-0.30 | 0.01-0.15 |
| SRF* (Hz) | 0.50-2.0 | 0.35-3.75 | 0.40-1.50 |

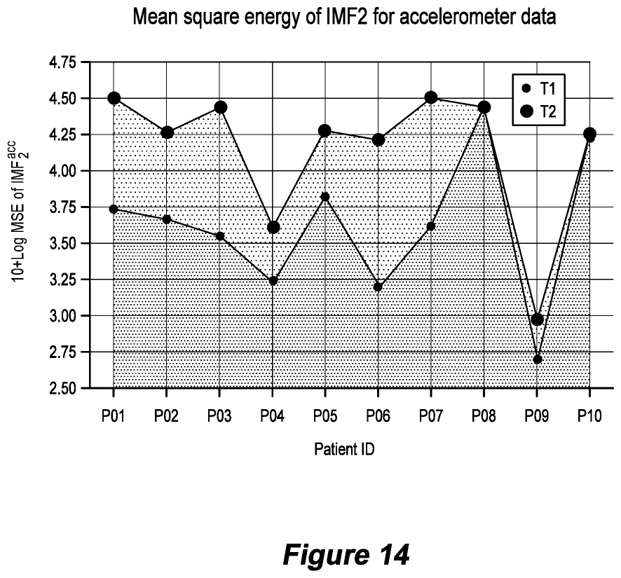

In the time-frequency domain, the mean square energy (MSE) of all the 6 IMFs was calculated using the empirical mode decomposition of the three measures (acc, gyr and pr). The MSE of IMF₂ of each subject obtained from acceleration (MSE_{IMF}_{₂}^{acc}) increased from T1 to T2 indicating progression of disease. This is illustrated in Table 10 as well as FIG. 14 which shows graphically the MSE of IMF₂ obtained from acceleration data from all subjects.

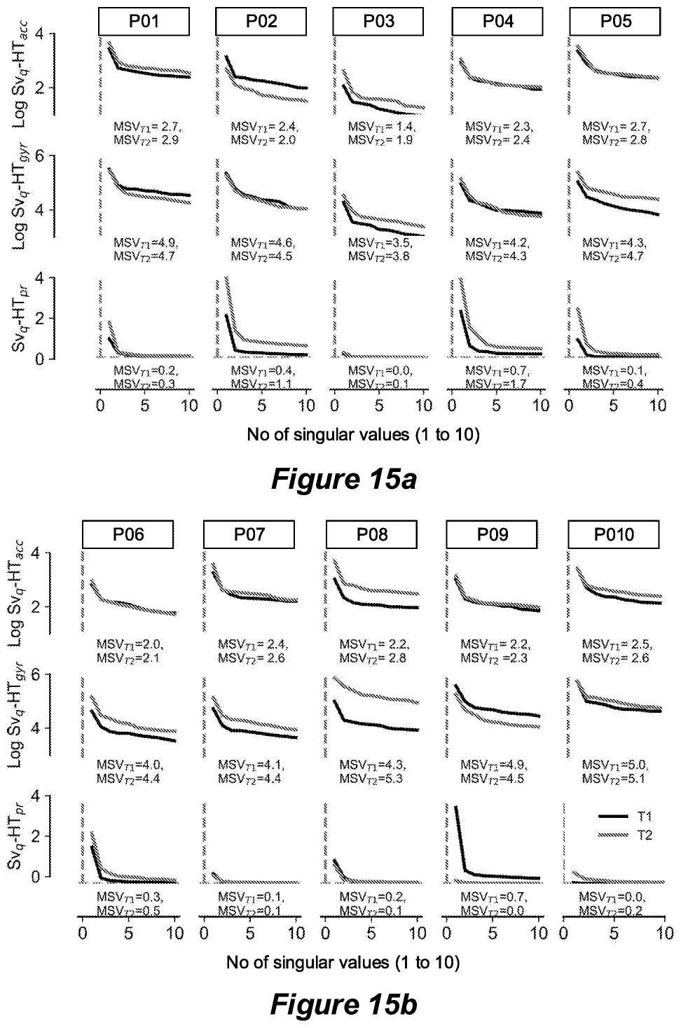

The initial singular values of the HHT matrix contained most of the matrix information and were considered features related to the energy. The first three singular values for Hilbert spectra of absolute acceleration were significantly higher at T2 than at T1 (Table 11). The remaining singular values did not demonstrate noticeable change from T1 to T2. In particular, the first singular value of the Hilbert spectra of acceleration (Sᵥ₁−HT_{acc}) for all participants was higher at T2 than at T1. This is illustrated in FIGS. 15A and 15B which show graphically the first ten singular values obtained using SvHT approach for the three measures (acc, gyr, pr) for patients P01 to P10. The mean singular value of the Hilbert spectra of pressure (Sᵥ_{_{1,2,3}}−HTₚᵣ) was also higher at T2 than at T1. However, for most participants, singular values of the Hilbert spectra for angular velocity (Sᵥ_{_{q}}−HT_{gyr}) were similar at both time points.

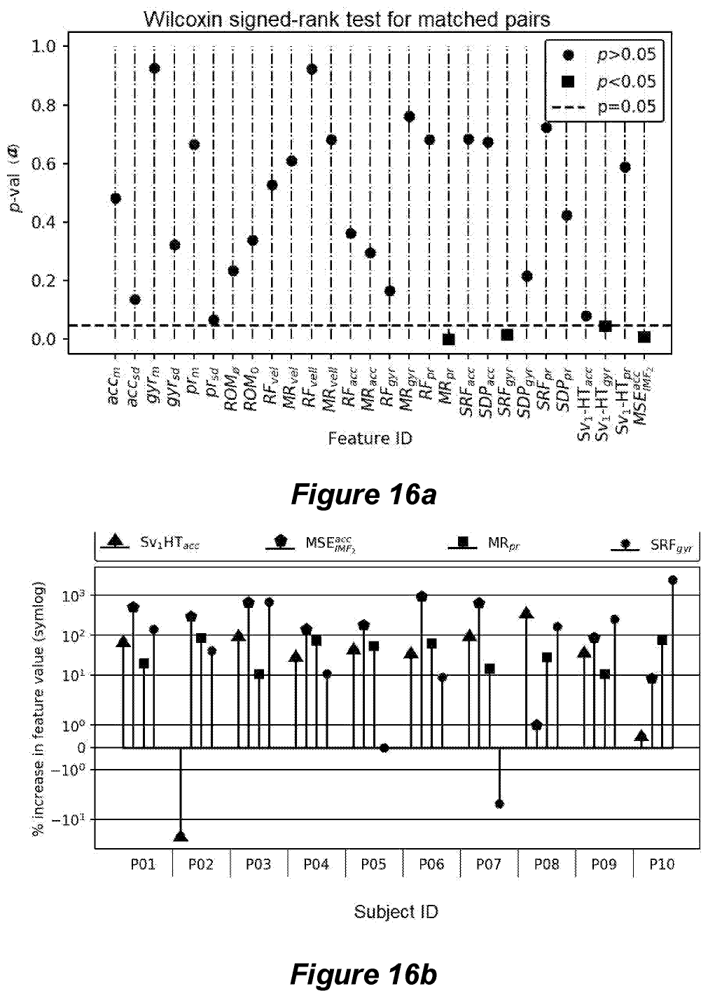

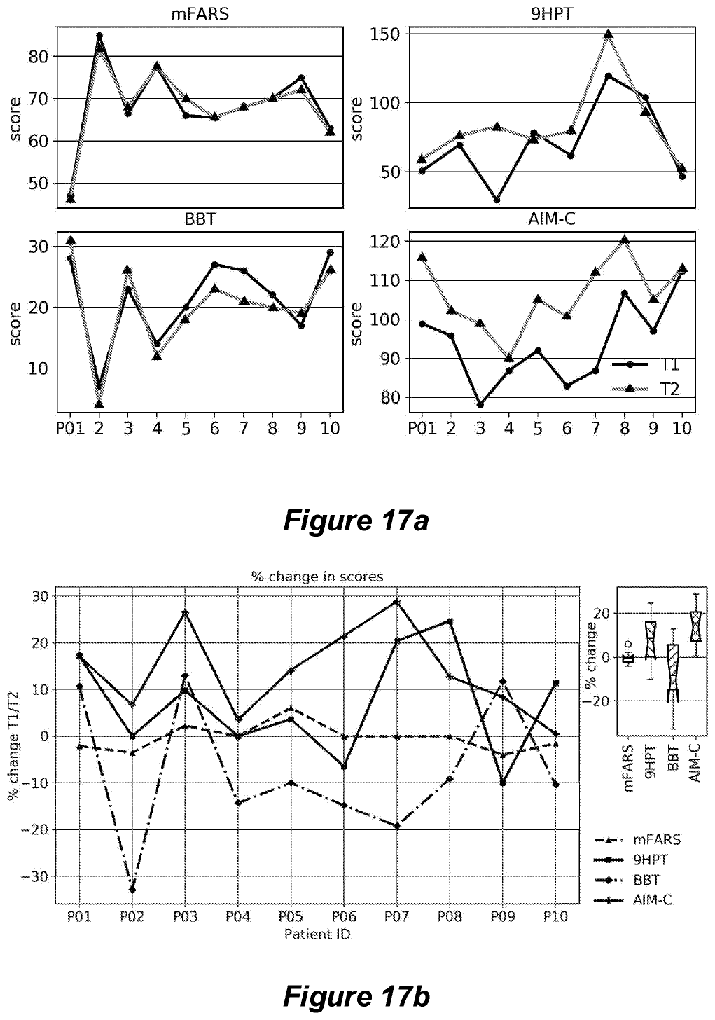

B) Comparison of clinical score versus AIM-C score for progression: The features whose values at T2 had significantly changed from their values at T1 are shown in FIG. 16(a). The feature values of MRₚᵣ, SRF_{gyr}, MSE_{IMF}_{₂}^{acc} and Sᵥ_{₁}−HT_{gyr} at T2 were significantly different, and higher than their values at T1. FIG. 16(b) shows the percentage change in feature values at T2 for these 4 features. These features therefore met the criteria for the “best feature set”. These features were combined using PCA and in a step 906 the AIM-C score for progression was generated by the first principal component based linear regression with the assumption that severity of ataxia increases from T1 to T2. The score was compared with mFARS, 9HPT, and BBT and the comparisons are represented graphically in FIG. 17(a) which compares clinical scores against the AIM-C score for progression in terms of longitudinal progression, and FIG. 17(b) which shows the comparison in terms of percentage change. In some instances, changes in the AIM-C score for progression between T1 to T2 are greater than the changes in clinical tests over the same period, suggesting

TABLE 10

| | | IMF1 | | IMF2 | | IMF3 | |
|---|---|---|---|---|---|---|---|
| | | T1 | T2 | T1 | T2 | T1 | T2 |
| acc | P003 | 3.35E−06 | 1.60E−08 | 5.32E−07 | 3.12E−06 | 1.42E−05 | 4.05E−06 |
| | P011 | 1.18E−07 | 8.11E−08 | 4.55E−07 | 1.81E−06 | 1.97E−07 | 8.88E−08 |
| | P022 | 9.03E−08 | 4.97E−07 | 3.51E−07 | 2.73E−06 | 6.70E−07 | 1.68E−05 |
| | P025 | 1.46E−07 | 1.23E−07 | 1.73E−07 | 4.09E−07 | 1.83E−06 | 2.35E−06 |
| | P026 | 1.55E−07 | 8.67E−07 | 6.58E−07 | 1.84E−06 | 1.92E−06 | 1.16E−07 |
| | P032 | 1.97E−08 | 1.49E−07 | 1.55E−07 | 1.63E−06 | 2.17E−06 | 7.28E−07 |
| | P033 | 1.20E−07 | 5.63E−07 | 4.11E−07 | 3.12E−06 | 4.70E−06 | 2.43E−05 |
| | P034 | 4.54E−07 | 7.02E−07 | 2.68E−06 | 2.72E−06 | 1.87E−08 | 9.59E−05 |
| | P035 | 5.32E−07 | 5.59E−07 | 5.00E−08 | 9.13E−08 | 8.50E−06 | 1.32E−06 |
| | P036 | 7.46E−07 | 4.74E−07 | 1.65E−06 | 1.78E−06 | 1.70E−04 | 1.76E−05 |
| gyr | P003 | 0.000262 | 7.08E−05 | 0.000163 | 0.000573 | 0.000239 | 0.000772 |
| | P011 | 6.78E−05 | 2.04E−05 | 4.04E−05 | 0.000163 | 7.70E−05 | 5.98E−05 |
| | P022 | 1.05E−05 | 3.41E−05 | 2.70E−05 | 4.04E−05 | 2.71E−05 | 7.67E−05 |
| | P025 | 7.94E−06 | 3.46E−05 | 2.44E−06 | 2.70E−05 | 3.26E−05 | 1.88E−05 |
| | P026 | 2.45E−05 | 4.61E−06 | 0.000152 | 2.44E−06 | 0.000436 | 0.000233 |
| | P032 | 5.88E−06 | 1.02E−05 | 1.11E−05 | 0.000152 | 2.52E−05 | 0.000227 |
| | P033 | 3.90E−06 | 1.51E−06 | 5.76E−05 | 1.11E−05 | 0.000406 | 7.04E−06 |
| | P034 | 8.82E−05 | 0.001563 | 0.000107 | 5.76E−05 | 5.00E−06 | 4.08E−05 |
| | P035 | 0.000108 | 0.000103 | 0.000687 | 0.000107 | 0.000222 | 0.000132 |
| | P036 | 0.000505 | 7.70E−06 | 0.000384 | 0.000687 | 0.002273 | 0.000248 |
| pr | P003 | 1.03E−07 | 1.08E−09 | 5.22E−07 | 9.40E−07 | 9.48E−07 | 9.38E−07 |
| | P011 | 7.52E−11 | 1.64E−10 | 5.80E−11 | 1.93E−09 | 1.17E−08 | 2.83E−08 |
| | P022 | 1.56E−09 | 2.54E−08 | 1.49E−08 | 1.71E−07 | 1.02E−07 | 3.73E−07 |
| | P025 | 3.09E−09 | 2.47E−09 | 2.99E−08 | 1.19E−07 | 5.45E−07 | 2.34E−06 |
| | P026 | 9.50E−10 | 2.78E−09 | 8.76E−07 | 1.36E−07 | 6.40E−07 | 1.35E−06 |
| | P032 | 2.16E−08 | 2.77E−09 | 1.43E−06 | 6.76E−08 | 2.04E−06 | 9.86E−08 |
| | P033 | 1.53E−09 | 1.59E−10 | 1.05E−07 | 1.44E−07 | 5.44E−07 | 1.40E−07 |
| | P034 | 6.00E−08 | 2.35E−08 | 4.79E−08 | 5.36E−07 | 6.72E−07 | 4.74E−07 |
| | P035 | 9.56E−09 | 7.98E−09 | 1.53E−07 | 3.82E−08 | 5.47E−07 | 2.30E−08 |
| | P036 | 9.35E−10 | 1.97E−09 | 1.94E−07 | 9.43E−08 | 1.59E−07 | 9.19E−07 |

| | | IMF4 | | IMF5 | | IMF6 | |
|---|---|---|---|---|---|---|---|
| | | T1 | T2 | T1 | T2 | T1 | T2 |
| acc | P003 | 9.24E−06 | 8.29E−06 | 1.64E−05 | 1.58E−05 | 3.25E−05 | 4.14E−06 |
| | P011 | 1.28E−06 | 2.70E−06 | 5.09E−07 | 2.47E−06 | 2.96E−06 | 2.34E−06 |
| | P022 | 3.95E−06 | 2.30E−05 | 1.33E−05 | 4.77E−05 | 8.22E−06 | 1.03E−04 |
| | P025 | 5.44E−06 | 1.45E−06 | 4.63E−07 | 5.86E−06 | 1.84E−05 | 1.10E−05 |
| | P026 | 1.81E−05 | 9.98E−06 | 4.11E−05 | 7.18E−06 | 2.21E−06 | 6.61E−06 |
| | P032 | 3.09E−05 | 4.50E−05 | 1.74E−07 | 7.13E−06 | 3.23E−06 | 2.47E−05 |
| | P033 | 1.30E−06 | 2.90E−05 | 2.99E−05 | 6.84E−05 | 2.12E−05 | 4.12E−05 |
| | P034 | 2.95E−05 | 3.19E−05 | 4.40E−05 | 1.86E−05 | 2.69E−05 | 9.27E−06 |
| | P035 | 2.88E−05 | 6.61E−05 | 5.10E−05 | 6.89E−05 | 1.40E−05 | 2.46E−05 |
| | P036 | 1.89E−05 | 4.97E−05 | 8.78E−06 | 1.62E−04 | 2.37E−05 | 3.61E−05 |
| gyr | P003 | 0.000217 | 0.000231 | 0.000388 | 4.70E−05 | 0.000126 | 0.00017 |
| | P011 | 9.83E−06 | 7.50E−05 | 2.18E−05 | 3.93E−05 | 9.55E−05 | 2.30E−05 |
| | P022 | 0.000103 | 0.000508 | 0.000118 | 0.000211 | 6.84E−05 | 0.000326 |
| | P025 | 2.99E−05 | 1.38E−05 | 9.23E−06 | 0.000492 | 2.92E−05 | 9.77E−05 |
| | P026 | 0.000274 | 0.000252 | 0.000774 | 0.000127 | 0.000469 | 0.000189 |
| | P032 | 0.000108 | 0.000128 | 7.17E−05 | 0.000452 | 0.000233 | 7.48E−05 |
| | P033 | 2.53E−05 | 0.00033 | 0.000252 | 0.000633 | 1.23E−05 | 0.000289 |
| | P034 | 0.000175 | 0.000675 | 0.000148 | 0.000835 | 0.000127 | 0.001961 |
| | P035 | 1.93E−06 | 9.74E−05 | 5.01E−05 | 9.17E−05 | 9.93E−05 | 7.86E−06 |
| | P036 | 0.002482 | 9.83E−05 | 0.002118 | 0.00091 | 0.000839 | 0.001472 |
| pr | P003 | 5.25E−07 | 1.86E−06 | 2.28E−07 | 1.51E−06 | 5.22E−07 | 1.51E−06 |
| | P011 | 1.62E−07 | 3.13E−07 | 1.03E−07 | 3.14E−08 | 2.76E−07 | 1.95E−06 |
| | P022 | 2.63E−07 | 4.15E−07 | 5.96E−08 | 7.62E−07 | 7.52E−08 | 5.12E−07 |
| | P025 | 5.58E−07 | 2.50E−07 | 5.35E−07 | 1.93E−06 | 3.45E−06 | 5.18E−06 |
| | P026 | 3.76E−07 | 1.04E−06 | 5.66E−07 | 3.65E−07 | 4.77E−07 | 1.35E−06 |
| | P032 | 6.36E−07 | 2.29E−06 | 1.67E−07 | 4.97E−07 | 5.51E−06 | 9.91E−07 |
| | P033 | 8.35E−07 | 1.48E−06 | 1.65E−08 | 1.85E−07 | 2.74E−07 | 3.13E−07 |
| | P034 | 3.01E−06 | 8.95E−07 | 8.32E−07 | 8.09E−07 | 5.99E−07 | 1.86E−07 |
| | P035 | 7.38E−07 | 3.45E−09 | 2.14E−06 | 6.83E−07 | 4.18E−07 | 3.28E−08 |
| | P036 | 6.27E−08 | 1.73E−06 | 3.23E−08 | 1.62E−06 | 1.94E−07 | 9.43E−08 |

TABLE 11

| | acc (log) | | | | | | gyr (log) | | | | | | pr | | | | | |
|---|---|---|---|---|---|---|---|---|---|---|---|---|---|---|---|---|---|---|
| | sv1 | | sv2 | | sv3 | | sv1 | | sv2 | | sv3 | | sv1 | | sv2 | | sv3 | |
| | T1 | T2 | T1 | T2 | T1 | T2 | T1 | T2 | T1 | T2 | T1 | T2 | T1 | T2 | T1 | T2 | T1 | T2 |
| P003 | 3.42 | 3.64 | 2.71 | 2.94 | 2.64 | 2.80 | 5.47 | 5.44 | 4.87 | 4.79 | 5.47 | 4.56 | 0.94 | 1.75 | 0.23 | 0.28 | 0.15 | 0.20 |
| P011 | 3.13 | 3.00 | 2.38 | 2.10 | 2.36 | 1.95 | 5.33 | 5.24 | 4.76 | 4.68 | 5.33 | 4.68 | 2.08 | 4.32 | 0.37 | 1.36 | 0.28 | 0.89 |
| P022 | 2.33 | 2.61 | 1.46 | 1.84 | 1.40 | 1.61 | 4.24 | 4.50 | 3.51 | 3.91 | 4.24 | 3.91 | 0.19 | 0.25 | 0.06 | 0.06 | 0.02 | 0.05 |
| P025 | 2.93 | 3.04 | 2.36 | 2.37 | 2.24 | 2.32 | 4.93 | 5.11 | 4.31 | 4.49 | 4.93 | 4.49 | 4.28 | 10.85 | 0.59 | 1.49 | 0.32 | 1.02 |
| P026 | 3.35 | 3.51 | 2.86 | 3.01 | 2.63 | 2.58 | 5.00 | 5.35 | 4.44 | 4.78 | 5.00 | 4.78 | 0.89 | 2.41 | 0.14 | 0.65 | 0.05 | 0.33 |
| P032 | 2.68 | 2.81 | 2.15 | 2.27 | 2.06 | 2.21 | 4.66 | 5.17 | 4.11 | 4.51 | 4.66 | 4.51 | 1.74 | 2.40 | 0.27 | 0.71 | 0.14 | 0.48 |
| P033 | 3.10 | 3.39 | 2.51 | 2.48 | 2.31 | 2.42 | 4.76 | 5.15 | 4.15 | 4.52 | 4.76 | 4.52 | 0.48 | 0.37 | 0.06 | 0.11 | 0.03 | 0.07 |
| P034 | 2.87 | 3.52 | 2.20 | 2.71 | 2.04 | 2.65 | 5.02 | 5.84 | 4.34 | 5.55 | 5.02 | 5.55 | 1.09 | 0.87 | 0.29 | 0.17 | 0.11 | 0.14 |
| P035 | 2.86 | 3.00 | 2.16 | 2.24 | 2.06 | 2.12 | 5.57 | 5.30 | 5.00 | 4.70 | 5.57 | 4.70 | 3.63 | 0.13 | 0.62 | 0.04 | 0.43 | 0.03 |
| P036 | 3.27 | 3.27 | 2.57 | 2.66 | 2.36 | 2.55 | 5.74 | 5.73 | 5.01 | 5.18 | 5.74 | 5.18 | 0.04 | 0.53 | 0.01 | 0.25 | 0.00 | 0.18 |

that the AIM-C score for progression is more sensitive to disease progression than the standard clinical tests.

C) Performance of AIM-C score for progression compared to clinical scores over time: The sensitivity of the AIM-C score for progression from T1 to T2 was expressed in terms of effect size (d) as shown in Table 12, where d is a dependent p-value obtained using Wilcoxon signed rank test for matched pairs (*d_{0.8} and d_{0.9} refer to the effect size for achieving 80% and 90% power (Pa) respectively). The effect-size for each test was such that AIM-C (1.59)>NHPT (1.23)>BBT (0.633)>mFARS (0.00) when then power (Pα) was set at 80%.

TABLE 12

| | mFARS | 9HPT | BBT | AIM-C |
|---|---|---|---|---|
| No. of samples | 10 | 8 | 10 | 10 |
| p-value | 0.675 | 0.007 | 0.257 | 0.002 |
| d_{0.8} * | 0.00 | 1.23 | 0.470 | 1.59 |
| d_{0.9} * | 0.04 | 1.45 | 0.633 | 1.79 |

The scores were further compared using multivariate analysis by post-hoc tests using Holm-Bonferonni method for non-normal data distributions (FWER+0.05, alphaSi=0.01, alphaBonf=0.008). Table 13 presents the comparison of scores and shows significant difference between AIM-C scores for progression and other clinical scales (p-value=0.0 indicates highly significant values). The performance of AIM-C score for progression was significantly different (p=0.000) from the other clinical scores in T1 and T2. The BBT score was also significantly different from the other scores. The 9HPT score was not significantly different (p=0.104) to the mFARS score in capturing the change in upper limb function over time but noticeably different to the AIM-C and BBT scores.

TABLE 13

| group 1 | group 2 | stat | p-value | pval-corr |
|---|---|---|---|---|
| AIM-C | BBT | 24.51 | 0.000 | 0.000 |
| AIM-C | 9HPT | 3.51 | 0.003 | 0.006 |
| AIM-C | mFARS | 9.37 | 0.000 | 0.000 |
| BBT | 9HPT | −6.65 | 0.000 | 0.000 |
| BBT | mFARS | −14.47 | 0.000 | 0.000 |
| 9HPT | mFARS | 1.73 | 0.104 | 0.1036 |

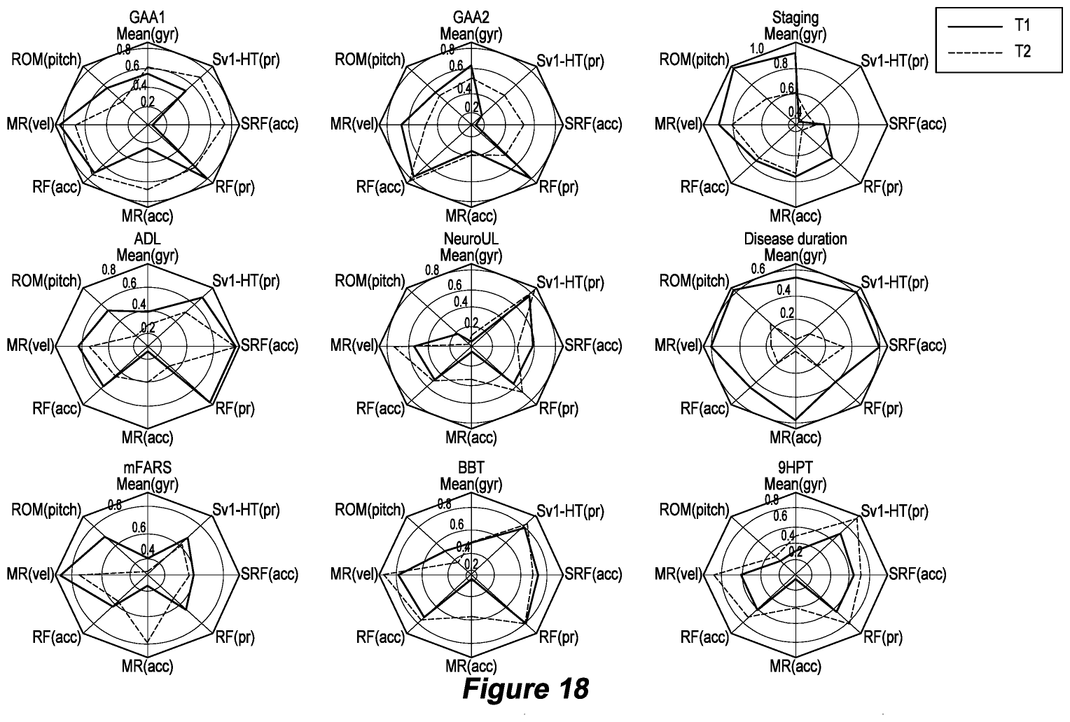

D) Correlation between T1 and T2 values of extracted features and clinical scores. Clinical measures (mFARS score, Neuro UL, Functional Staging of Ataxia score and the ADL score) and fixed clinical parameters (GAA1 and GAA2) significantly correlated with the kinematic and kinetic features as shown in FIG. 18 and Table 14.

The time domain feature ROM_{θ}, highly correlated (P^{T1}=0.99, P^{T2}=0.7) with the Functional Staging of Ataxia. The mean angular velocity (gyrₘ) also correlated with the Functional Staging of Ataxia Score at T1.

The frequency domain feature MRᵥₑₗ correlated highest with GAA1 (P^{T1}=0.84, P^{T2}=0.70), the Functional Staging of Ataxia (P_{T1}=0.91, P^{T2}=0.82) and the mFARS score (P^{T1}=0.85, P^{T2}=0.72). RFₚᵣ highly correlated (P^{T1}>0.75) with the clinical scores (GAA1, GAA2, ADL) at T1, but not at T2. The kinematic feature SRF_{Q}cc correlated strongest (P^{T1}=0.78, P^{T2}=0.80) at T1 and T2 with the ADL scores. Most of the features correlated (P_{C}^{T1}>0.8) with the Functional Staging of Ataxia.

The highest correlation (P_{C}^{T1}=0.76, P_{C}^{T2}=0.82) obtained for (NeuroUL) upper limb scores was with the Sᵥ₁-HTₚᵣ feature. Sᵥ₂-HTₚᵣ also correlated the highest (P_{C}^{T1}=0.83, P_{C}^{T2}=0.95) with traditional measures of upper limb function (the 9HPT and BBT). The feature correlation obtained for disease duration was not significant (P_{c}<0.5) at T2. For detailed reporting, the statistical significance (p<0.01) of features that significantly correlated with the clinical scores are provided in Table 14.

### Discussion

Example 2 demonstrates that there were extractable features whose values significantly changed (α<0.05) over the duration of this study for most participants and that when these features were represented in the AIM-C score for progression, their statistical power was greater than conventional clinical tests. This finding implies that there is information in the kinetic and kinematic data that is relevant to FRDA and in particularly to its progression which cannot be determined using clinical tests alone, pointing to the utility of instrumented assessments as disclosed herein.

TABLE 14

| | GAA1 | | | | GAA2 | | | | Staging | | | |
|---|---|---|---|---|---|---|---|---|---|---|---|---|
| Features | T1 | p | T2 | p | T1 | p | T2 | p | T1 | p | T2 | p |

Time features

| | | | | | | | | | | | | |
|---|---|---|---|---|---|---|---|---|---|---|---|---|
| Mean acceleration | 0.23 | 0.53 | 0.60 | 0.07 | 0.32 | 0.37 | 0.36 | 0.31 | 0.67 | 0.03 | 0.59 | 0.07 |
| Standard deviation of acc | 0.18 | 0.62 | 0.66 | 0.04 | 0.36 | 0.31 | 0.57 | 0.09 | 0.69 | 0.03 | 0.66 | 0.04 |
| Mean angular velocity | 0.54 | 0.11 | 0.71 | 0.02 | 0.65 | 0.04 | 0.54 | 0.11 | 0.92 | 0.00 | 0.62 | 0.05 |
| Standard deviation of gyr | 0.46 | 0.18 | 0.63 | 0.05 | 0.62 | 0.06 | 0.59 | 0.07 | 0.87 | 0.00 | 0.56 | 0.09 |
| Mean differential pressure | 0.29 | 0.42 | 0.68 | 0.03 | 0.47 | 0.18 | 0.56 | 0.09 | 0.24 | 0.50 | 0.38 | 0.27 |
| Standard deviation of pr | 0.37 | 0.29 | 0.33 | 0.35 | 0.37 | 0.29 | 0.20 | 0.57 | 0.39 | 0.26 | 0.25 | 0.49 |
| ROM of roll angle | 0.70 | 0.02 | 0.58 | 0.08 | 0.42 | 0.23 | 0.30 | 0.40 | 0.46 | 0.18 | 0.62 | 0.06 |
| ROM of pitch angle | 0.55 | 0.10 | 0.51 | 0.13 | 0.53 | 0.12 | 0.50 | 0.15 | 0.99 | 0.00 | 0.66 | 0.04 |

Frequency features

| | | | | | | | | | | | | |
|---|---|---|---|---|---|---|---|---|---|---|---|---|
| RF of velocity | 0.11 | 0.76 | 0.29 | 0.42 | 0.49 | 0.15 | 0.02 | 0.97 | 0.16 | 0.65 | 0.16 | 0.67 |
| MR of velocity | 0.48 | 0.16 | 0.76 | 0.01 | 0.66 | 0.04 | 0.55 | 0.10 | 0.88 | 0.00 | 0.83 | 0.00 |
| RF of linear velocity | 0.44 | 0.21 | 0.48 | 0.16 | 0.31 | 0.39 | 0.01 | 0.97 | 0.32 | 0.37 | 0.59 | 0.07 |
| MR of linear velocity | 0.82 | 0.00 | 0.76 | 0.01 | 0.68 | 0.03 | 0.48 | 0.16 | 0.90 | 0.00 | 0.57 | 0.08 |
| RF of acceleration | 0.70 | 0.02 | 0.78 | 0.01 | 0.78 | 0.01 | 0.82 | 0.00 | 0.76 | 0.01 | 0.75 | 0.01 |
| MR of acceleration | 0.24 | 0.50 | 0.77 | 0.01 | 0.34 | 0.33 | 0.38 | 0.28 | 0.77 | 0.01 | 0.74 | 0.01 |
| RF of angular velocity | 0.63 | 0.05 | 0.70 | 0.02 | 0.77 | 0.01 | 0.63 | 0.05 | 0.82 | 0.00 | 0.63 | 0.05 |
| MR of angular velocity | 0.08 | 0.83 | 0.47 | 0.17 | 0.05 | 0.89 | 0.30 | 0.41 | 0.18 | 0.62 | 0.37 | 0.29 |
| RF of pressure | 0.79 | 0.01 | 0.74 | 0.02 | 0.78 | 0.01 | 0.50 | 0.14 | 0.73 | 0.02 | 0.50 | 0.14 |
| MR of pressure | 0.30 | 0.40 | 0.40 | 0.25 | 0.36 | 0.31 | 0.32 | 0.37 | 0.32 | 0.37 | 0.30 | 0.40 |
| Frequency of SDP of acc | 0.50 | 0.14 | 0.47 | 0.17 | 0.59 | 0.07 | 0.34 | 0.34 | 0.54 | 0.11 | 0.52 | 0.13 |
| Second peak of acc | 0.06 | 0.87 | 0.79 | 0.01 | 0.04 | 0.90 | 0.54 | 0.11 | 0.56 | 0.10 | 0.50 | 0.14 |
| Frequency of SDP of gyr | 0.30 | 0.41 | 0.64 | 0.05 | 0.15 | 0.68 | 0.36 | 0.30 | 0.28 | 0.43 | 0.46 | 0.18 |
| Second peak of gyr | 0.36 | 0.30 | 0.09 | 0.81 | 0.72 | 0.02 | 0.12 | 0.74 | 0.80 | 0.01 | 0.05 | 0.88 |
| Frequency of SDP of pr | 0.71 | 0.02 | 0.69 | 0.03 | 0.82 | 0.00 | 0.58 | 0.08 | 0.76 | 0.01 | 0.77 | 0.01 |
| Second peak of pressure | 0.31 | 0.38 | 0.23 | 0.52 | 0.20 | 0.58 | 0.13 | 0.71 | 0.39 | 0.26 | 0.10 | 0.77 |
| Euler RF of roll angle | 0.82 | 0.00 | 0.52 | 0.12 | 0.65 | 0.04 | 0.57 | 0.08 | 0.92 | 0.00 | 0.49 | 0.15 |
| Euler MR of roll angle | 0.29 | 0.41 | 0.44 | 0.20 | 0.39 | 0.27 | 0.27 | 0.46 | 0.47 | 0.17 | 0.57 | 0.09 |
| Euler RF of pitch angle | 0.52 | 0.12 | 0.53 | 0.11 | 0.76 | 0.01 | 0.46 | 0.18 | 0.70 | 0.02 | 0.25 | 0.48 |
| Euler MR of pitch angle | 0.23 | 0.53 | 0.63 | 0.05 | 0.39 | 0.27 | 0.49 | 0.15 | 0.56 | 0.09 | 0.78 | 0.01 |
| Euler RF of yaw angle | 0.54 | 0.11 | 0.50 | 0.14 | 0.51 | 0.13 | 0.40 | 0.25 | 0.58 | 0.08 | 0.29 | 0.41 |
| Euler MR of yaw angle | 0.18 | 0.63 | 0.51 | 0.13 | 0.25 | 0.49 | 0.36 | 0.31 | 0.16 | 0.66 | 0.74 | 0.01 |

Time-Frequency features

| | | | | | | | | | | | | |
|---|---|---|---|---|---|---|---|---|---|---|---|---|
| SVD-HHT of acc | 0.25 | 0.48 | 0.41 | 0.24 | 0.52 | 0.12 | 0.36 | 0.31 | 0.72 | 0.02 | 0.35 | 0.32 |
| SVD-HHT of gyr | 0.29 | 0.41 | 0.60 | 0.07 | 0.15 | 0.67 | 0.19 | 0.59 | 0.65 | 0.04 | 0.53 | 0.11 |
| SVD-HHT of pr | 0.43 | 0.21 | 0.70 | 0.02 | 0.14 | 0.71 | 0.45 | 0.19 | 0.34 | 0.33 | 0.45 | 0.19 |
| MSE of IMF2 of acc | 0.59 | 0.07 | 0.00 | 0.99 | 0.04 | 0.90 | 0.07 | 0.85 | 0.48 | 0.16 | 0.18 | 0.62 |

Traditional Tests

| | | | | | | | | | | | | |
|---|---|---|---|---|---|---|---|---|---|---|---|---|
| Nine Hole Peg Test | 0.89 | 0.00 | 0.86 | 0.00 | 0.58 | 0.08 | 0.54 | 0.10 | 0.54 | 0.11 | 0.76 | 0.01 |
| Box and Block Test | 0.72 | 0.02 | 0.73 | 0.02 | 0.35 | 0.32 | 0.36 | 0.30 | 0.29 | 0.42 | 0.49 | 0.15 |

| | ADL | | | | Neuro UL | | | | mFARS | | BBT | | 9HPT | |
|---|---|---|---|---|---|---|---|---|---|---|---|---|---|---|
| Features | T1 | p | T2 | p | T1 | p | T2 | p | T1 | T2 | T1 | T2 | T1 | T2 |

Time features

| | | | | | | | | | | | | | | |
|---|---|---|---|---|---|---|---|---|---|---|---|---|---|---|
| Mean acceleration | 0.00 | 0.99 | 0.10 | 0.78 | 0.10 | 0.77 | 0.34 | 0.33 | 0.34 | 0.58 | 0.18 | 0.37 | 0.13 | 0.40 |
| Standard deviation of acc | 0.07 | 0.84 | 0.12 | 0.73 | 0.04 | 0.92 | 0.25 | 0.48 | 0.27 | 0.52 | 0.06 | 0.36 | 0.00 | 0.35 |
| Mean angular velocity | 0.40 | 0.25 | 0.29 | 0.41 | 0.14 | 0.70 | 0.37 | 0.29 | 0.53 | 0.52 | 0.47 | 0.49 | 0.32 | 0.55 |
| Standard deviation of gyr | 0.27 | 0.45 | 0.23 | 0.53 | 0.01 | 0.97 | 0.24 | 0.51 | 0.41 | 0.37 | 0.31 | 0.38 | 0.22 | 0.47 |
| Mean differential pressure | 0.64 | 0.05 | 0.43 | 0.21 | 0.31 | 0.38 | 0.62 | 0.05 | 0.19 | 0.44 | 0.56 | 0.74 | 0.45 | 0.69 |
| Standard deviation of pr | 0.73 | 0.02 | 0.50 | 0.14 | 0.50 | 0.14 | 0.56 | 0.10 | 0.43 | 0.34 | 0.68 | 0.60 | 0.62 | 0.76 |
| ROM of roll angle | 0.33 | 0.35 | 0.65 | 0.04 | 0.62 | 0.06 | 0.39 | 0.26 | 0.59 | 0.65 | 0.69 | 0.53 | 0.54 | 0.44 |
| ROM of pitch angle | 0.53 | 0.11 | 0.37 | 0.29 | 0.15 | 0.68 | 0.17 | 0.64 | 0.68 | 0.45 | 0.49 | 0.37 | 0.27 | 0.39 |

Frequency features

| | | | | | | | | | | | | | | |
|---|---|---|---|---|---|---|---|---|---|---|---|---|---|---|
| RF of velocity | 0.40 | 0.25 | 0.07 | 0.85 | 0.16 | 0.67 | 0.01 | 0.98 | 0.12 | 0.21 | 0.21 | 0.03 | 0.30 | 0.08 |
| MR of velocity | 0.35 | 0.33 | 0.44 | 0.20 | 0.18 | 0.62 | 0.38 | 0.28 | 0.50 | 0.76 | 0.45 | 0.59 | 0.29 | 0.50 |
| RF of linear velocity | 0.15 | 0.67 | 0.60 | 0.06 | 0.10 | 0.79 | 0.43 | 0.21 | 0.22 | 0.83 | 0.30 | 0.61 | 0.24 | 0.29 |
| MR of linear velocity | 0.63 | 0.05 | 0.66 | 0.04 | 0.53 | 0.12 | 0.85 | 0.00 | 0.84 | 0.70 | 0.78 | 0.70 | 0.60 | 0.70 |
| RF of acceleration | 0.57 | 0.08 | 0.52 | 0.12 | 0.46 | 0.18 | 0.57 | 0.09 | 0.61 | 0.59 | 0.73 | 0.75 | 0.60 | 0.75 |
| MR of acceleration | 0.12 | 0.73 | 0.41 | 0.24 | 0.02 | 0.96 | 0.44 | 0.20 | 0.36 | 0.80 | 0.21 | 0.57 | 0.08 | 0.48 |
| RF of angular velocity | 0.45 | 0.20 | 0.43 | 0.21 | 0.37 | 0.29 | 0.44 | 0.21 | 0.57 | 0.49 | 0.63 | 0.59 | 0.43 | 0.64 |
| MR of angular velocity | 0.05 | 0.89 | 0.23 | 0.53 | 0.35 | 0.31 | 0.29 | 0.41 | 0.05 | 0.48 | 0.15 | 0.45 | 0.08 | 0.28 |
| RF of pressure | 0.57 | 0.09 | 0.54 | 0.11 | 0.54 | 0.11 | 0.82 | 0.00 | 0.63 | 0.63 | 0.80 | 0.87 | 0.64 | 0.87 |
| MR of pressure | 0.71 | 0.02 | 0.51 | 0.13 | 0.39 | 0.26 | 0.57 | 0.09 | 0.30 | 0.36 | 0.62 | 0.64 | 0.55 | 0.78 |
| Frequency of SDP of acc | 0.77 | 0.01 | 0.77 | 0.01 | 0.55 | 0.10 | 0.53 | 0.11 | 0.58 | 0.52 | 0.71 | 0.71 | 0.64 | 0.76 |
| Second peak of acc | 0.27 | 0.45 | 0.12 | 0.75 | 0.40 | 0.25 | 0.56 | 0.09 | 0.02 | 0.51 | 0.23 | 0.56 | 0.29 | 0.57 |
| Frequency of SDP of gyr | 0.01 | 0.97 | 0.57 | 0.09 | 0.68 | 0.03 | 0.86 | 0.00 | 0.30 | 0.62 | 0.35 | 0.86 | 0.47 | 0.94 |
| Second peak of gyr | 0.34 | 0.34 | 0.16 | 0.66 | 0.07 | 0.85 | 0.41 | 0.25 | 0.25 | 0.13 | 0.33 | 0.26 | 0.15 | 0.27 |
| Frequency of SDP of pr | 0.54 | 0.11 | 0.82 | 0.00 | 0.44 | 0.20 | 0.64 | 0.05 | 0.58 | 0.73 | 0.73 | 0.88 | 0.57 | 0.81 |
| Second peak of pressure | 0.64 | 0.04 | 0.42 | 0.23 | 0.51 | 0.13 | 0.03 | 0.93 | 0.44 | 0.12 | 0.65 | 0.16 | 0.54 | 0.20 |
| Euler RF of roll angle | 0.66 | 0.04 | 0.37 | 0.29 | 0.54 | 0.11 | 0.49 | 0.15 | 0.87 | 0.42 | 0.79 | 0.64 | 0.60 | 0.69 |
| Euler MR of roll angle | 0.08 | 0.83 | 0.73 | 0.02 | 0.00 | 0.99 | 0.15 | 0.69 | 0.24 | 0.53 | 0.17 | 0.51 | 0.06 | 0.36 |
| Euler RF of pitch angle | 0.30 | 0.40 | 0.34 | 0.34 | 0.29 | 0.41 | 0.48 | 0.16 | 0.47 | 0.25 | 0.44 | 0.57 | 0.41 | 0.51 |
| Euler MR of pitch angle | 0.03 | 0.94 | 0.35 | 0.33 | 0.16 | 0.67 | 0.29 | 0.41 | 0.24 | 0.68 | 0.21 | 0.47 | 0.21 | 0.44 |
| Euler RF of yaw angle | 0.58 | 0.08 | 0.41 | 0.24 | 0.36 | 0.31 | 0.46 | 0.18 | 0.65 | 0.27 | 0.49 | 0.55 | 0.46 | 0.62 |
| Euler MR of yaw angle | 0.05 | 0.89 | 0.90 | 0.00 | 0.34 | 0.34 | 0.31 | 0.38 | 0.09 | 0.69 | 0.06 | 0.63 | 0.26 | 0.50 |

Time-Frequency features

| | | | | | | | | | | | | | | |
|---|---|---|---|---|---|---|---|---|---|---|---|---|---|---|
| SVD-HHT of acc | 0.19 | 0.59 | 0.15 | 0.69 | 0.23 | 0.53 | 0.01 | 0.97 | 0.17 | 0.17 | 0.13 | 0.16 | 0.12 | 0.22 |
| SVD-HHT of gyr | 0.04 | 0.91 | 0.28 | 0.43 | 0.18 | 0.62 | 0.39 | 0.27 | 0.51 | 0.63 | 0.19 | 0.44 | 0.14 | 0.41 |
| SVD-HHT of pr | 0.61 | 0.06 | 0.49 | 0.15 | 0.69 | 0.03 | 0.82 | 0.00 | 0.60 | 0.58 | 0.71 | 0.83 | 0.57 | 0.93 |
| MSE of IMF2 of acc | 0.31 | 0.38 | 0.15 | 0.67 | 0.26 | 0.47 | 0.22 | 0.54 | 0.67 | 0.07 | 0.37 | 0.23 | 0.27 | 0.25 |

Traditional Tests

| | | | | | | | | | | | | | | |
|---|---|---|---|---|---|---|---|---|---|---|---|---|---|---|
| Nine Hole Peg Test | 0.83 | 0.00 | 0.76 | 0.01 | 0.64 | 0.00 | 0.78 | 0.00 | 0.53 | 0.58 | 1.00 | 1.00 | 0.83 | 0.85 |
| Box and Block Test | 0.69 | 0.03 | 0.52 | 0.12 | 0.82 | 0.00 | 0.84 | 0.00 | 0.71 | 0.68 | 0.83 | 0.85 | 1.00 | 1.00 |

p refers to the p-value

Relevant features identified in Example 2 include the magnitude of pressure (MRₚᵣ) applied to the wall of the AIM-C device 200 while it was being held. People with ataxia have difficulty maintaining a constant grip force either as an impairment in predicting appropriate forces required for the grip and/or as a strategy for increasing mean grip force to avoid dropping objects during the execution of the task. As disease progresses, it is plausible that variability in grip force and the compensatory mean grip force may both increase, explaining why MRₚr was useful as a feature for monitoring disease progression.

Another feature identified in Example 2 as providing information about disease progression included the second peak of angular acceleration after application of FFT (SRF_{gyr}). This feature reveals rotational motion in axes not related to the primary axis of motion that may have arisen from dysmetria as well as instability at more proximal joints. Again it is plausible that this may have been due to increasing ataxia, making this feature a useful indicator of progression.

The Mean Square Energy of the second Intrinsic Mode Function obtained from acceleration (MSE_{IMF}_{₂}^{acc}), which is a feature from the time-frequency domain, is higher at T2 than at T1 in all participants and was the third feature selected for determining disease progression. It is possible that as ataxia worsens, the energy required to execute the task increases, thus reflecting worsening of disease.

The first singular values of the HHT spectrum of acceleration (Sᵥ₁−HT_{gyr}) was chosen as the fourth feature that showed significant difference between T1 and T2. Sᵥ₁−HT_{gyr} contained most of the time-frequency information, highlighting the inherent non-linear characteristics of the motion of the vessel during the task which are also likely to be more prominent as ataxia worsens.

Although the four features (MRₚᵣ, SRF_{gyr}, MSE_{IMF}_{₂}^{acc}, Sᵥ₁−HT_{gyr}) used to make the AIM-C score for progression were selected on a statistical basis, it also seems likely that they carry information about ataxic movement that could be expected to change as ataxia deteriorated. The relevance of the other features such as ROM_{θ}, MRᵥₑₗ, SRFacc, Sᵥ₁-HTₚᵣ to ataxic movement is supported by their correlation with clinical measures (mFARS score, NeuroUL, ADL) and clinical parameters (GAA1 and GAA2).

It is to be noted, however, that a larger sample of subjects with FRDA could yield a slightly different set of candidate features for the AIM-C score for progression. It is also possible that some of the four candidate features identified in Example 2 carry shared information (e.g. SRF_{gyr} and energy (Sᵥ₁-HT_{gyr})) although it is likely that such overlap has been addressed to at least some extent by PCA. Nevertheless, a larger population would permit a more detailed analysis with possibility of a more refined feature set to avoid overfitting. One of skill in the art could readily utilise the teachings of this disclosure in evaluating a larger dataset to determine such a refined feature set which is explicitly stated to be within the scope of this disclosure.

Irrespective of the dataset size utilised to identify the features of interest in Example 2, it is to be noted that the AIM-C score for progression has been demonstrated to have a greater effect size than the three clinical scores (mFARS, 9HPT and BBT). Therefore, the AIM-C score for progression disclosed herein is likely a more sensitive test for measuring disease progression than currently available clinical alternatives. A particular advantage of an instrumented form of assessment of disease progression with enhanced sensitivity as disclosed herein is that disease modifying therapies with small effects are more likely to be detected by the instrumented methods and systems disclosed herein, than by other measures, and therapies with larger effects could be identified in studies with a smaller sample size or shorter duration. This offers the potential to provide accurate, objective evaluation of novel therapies for ataxia, and to provide a tool to determine response to therapies which may be utilised in dosage titration.

The present disclosure provides an instrumented approach to measuring ataxia in the form of a device, such as a pressure-measuring canister, capable of sensing certain kinetic and kinematic parameters of interest to quantify the impairment levels of participants particularly when engaged in an activity that is closely associated with daily living. In particular, the functional task of simulated preparation for drinking can be utilised to capture characteristic features of disability manifestation in terms of diagnosis (separation of individuals with FA and controls) and severity assessment of individuals diagnosed with the debilitating condition of FA. Time and frequency domain analysis of these features enables the classification of individuals with FA and control subjects to reach an accuracy of 99% and a correlation level reaching 96% with the clinical scores. The instrumented approach to measuring ataxia also permits scoring of disease progression in an objective, repeatable manner that gives greater sensitivity than currently available clinical assessments of disease progression. Measuring disease progression is a useful proxy for testing the ability to measure disease modification. Because existing scales may not necessarily map onto functional capacity of daily activities, they may also neglect to capture the effect of emerging disease modifying therapies on factors impairing daily life. To date, longitudinal progression of FRDA using kinetic and kinematic sensing while simulating self-drinking has not been considered.

Unlike the 9HPT, which cannot be performed by severely impaired participants, assessments according to embodiments of the present disclosure can be performed by people who have significant motor deficits demonstrating the potential to add value to existing assessments available for use in clinical trials. In addition to the separation, severity and progression scoring capabilities, characterisation of movement dysfunction based on Holmesian dimensions (Stability, Timing delay, Accuracy, and Rhythmicity) provides clinically meaningful descriptions to the practitioner. For example, for the movement task of preparing for drinking, S.T.A.R. categorisation demonstrated ataxia manifestation predominantly in timing dimension. Indeed, using techniques disclosed herein, the ability to unravel and quantify the disability information is evident. Given the significant correlation with the clinical assessment scales, the present disclosure provides the basis for an objective assessment tool with simple and unique physical attributes vital for the use in the motor impairment management arena. Additionally, the device component is robust, compact and light weight and in embodiments where it resembles an object of daily living, it is readily taken up by subjects for assessment. Indeed these attributes and the relevance to ADL, suggest assessment in non-clinical settings allowing more regular and accurate testing to facilitate use in clinical trials as well as monitoring in homes and in community based rehabilitation programs.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that various modifications, additions and/or alterations may be made to the parts previously described without departing from the ambit of the present disclosure as defined in the claims appended hereto.

It is to be understood that the following claims are provided by way of example only, and are not intended to limit the scope of what may be claimed in the current or any future application. Features may be added to or omitted from the claims at a later date so as to further define or re-define the invention or inventions.

1-49. (canceled)

50. A movement monitoring system for objectively quantifying a motor control disorder in a subject, the system comprising:
(a) a movement detection device generating movement data representing movement of a limb of the subject, wherein the movement detection device comprises sensors measuring at least motion of the device and pressure applied to the device by the subject; and
(b) an analyser for analysing the movement data, the analyser comprising a processor and a memory containing code which, when executed by the processor:
(i) receives the movement data generated by the movement detection device;
(ii) applies the received movement data to an algorithmic model stored in the memory and identifies one or more features from the movement data that represent disordered movement by the subject; and
(ii) calculates from the one or more identified features a score corresponding to the existence of the motor control disorder in the subject.

51. The movement monitoring system according to claim 50, wherein the analyser applies the received movement data to one or more of:
(a) a first algorithmic model to identify a first set of features used by the processor to calculate a selection score which is indicative of presence or absence of the motor control disorder in the subject;
(b) a second algorithmic model to identify a second set of features used by the processor to calculate a severity score which is indicative of severity of the motor control disorder in the subject; and
(c) a third algorithmic model to identify a third set of features used by the processor to calculate a progression score which is indicative of progression of the motor control disorder in the subject.

52. The movement monitoring system according to claim 51, wherein the severity score calculated by the processor corresponds to a score obtained according to a clinical scale.

53. The movement monitoring system according to claim 51, wherein the first set of features used by the processor to calculate the selection score comprises one of the following feature sets:
(a) Pr_{RF}; or
(b) Pr_{RF}, A_{CC}_{_{RF}}^{X}, Gyro_{MR}^{XYZ}, Sₘ, Aₜ and Pr_{M}; and
(c) θ_{RF}^{c}, Acc_{RF}^{X}, Sₘ, Pr_{RF}, Aₜ, S^{T}, Pr_{M}, Ø_{RF}^{c} and Gyr_{MR}^{XYZ}.

54. The movement monitoring system according to claim 51, wherein the second set of features used by the processor to calculate the severity score is selected from a group comprising Pr_{RF} Pr_{M}, Aₜ and θ_{RF}^{c} and preferably comprises the feature set Pr_{M}, Aₜ and θ_{RF}^{c}.

55. The movement monitoring system according to claim 51, wherein the third set of features used by the processor to calculate the progression score is selected from a group comprising: (a) MRₚᵣ, SRF_{gyr}, MSE_{TMF}_{₂}^{acc}, Sᵥ₁-HT_{gyr}, ROM_{θ}, MRᵥₑₗ, SRF_{acc}, Sᵥ₁-HT_{gyr}; and preferably comprises MRₚᵣ, SRF_{gyr}, MSE_{TMF}_{₂}^{acc}, Sᵥ₁-HT_{gyr}.

56. The movement monitoring system according to claim 51, wherein the analyser categorises movement dysfunction in the subject by the processor calculating a contribution made by each of the first, second or third set of features to each of a plurality of movement characteristics that are attributable to movement dysfunction in the subject and optionally, wherein the plurality of movement characteristics correlate to clinically accepted descriptions of movement disorder and optionally, wherein the clinically accepted descriptions relate to one or more of stability, timing, accuracy and rhythmicity of the movement and optionally, wherein the analyser sums the contribution made by each of the features to each of the plurality of movement characteristics to determine a collective contribution to each of the plurality of movement characteristics.

57. The movement monitoring system according to claim 50, wherein the movement detection device simulates or is incorporated into an object of daily living and comprises one or more of:
(a) a pressure sensor;
(b) an accelerometer; and
(c) a gyroscope.

58. The movement monitoring system according to claim 50, wherein the movement detection device comprises a canister with a grasping portion and a pressure sensor for measuring pressure applied to the grasping portion by the subject.

59. The movement monitoring system according to claim 50, wherein the motor control disorder is spasticity, and features identified in the movement data that are used to indicate presence of spasticity include Pr_{SD} Pr_{RMS} Pr_{MR} Pr_{RF}.

60. A movement detection device for use with a system for objectively quantifying motor control disorder in a subject, the movement detection device comprising:
(a) a grasping portion; and
(b) a movement sensor comprising at least a pressure sensor generating pressure data representing pressure applied to the grasping portion and a motion sensor generating motion data representing movement of the device in multiple axes;
wherein the movement detection device simulates or is incorporated into an object of daily living.

61. The movement detection device according to claim 60, wherein the object of daily living is selected from a group comprising:
(a) a cup or drinking vessel;
(b) a spoon or eating utensil; and
(c) a brush or comb.

62. The movement detection device according to claim 60, comprising a canister simulating a cup or drinking vessel, the canister comprising a flexible body portion forming a fluid filled chamber and defining the grasping portion, and optionally
wherein the pressure sensor is a differential pressure sensor with a first input in fluid communication with the chamber and a second input in fluid communication with atmospheric pressure.

63. The movement detection device according to claim 60, comprising a one-way valve for releasable coupling with a fluid source to restore fluid pressure in the chamber.

64. The movement detection device according to claim 62, wherein the canister comprises a rigid base containing one or both of a microcontroller and a wireless communication module.

65. An automated method for objectively quantifying a motor control disorder in a subject, comprising the steps of:
(a) receiving at a processor movement data corresponding to movements of a limb of the subject, the movement data comprising at least pressure data and motion data;
(b) the processor applying the received movement data to an algorithmic model and identifying one or more features from the movement data that represent disordered movement in the subject;
(c) the processor calculating, from the one or more identified features, a score quantifying the motor control disorder in the subject; and
(d) the processor generating a display signal causing the calculated score to be presented on a display device.

66. The automated method of claim 65, wherein the processor applies the received movement data to one or more of:
(a) a first algorithmic model to identify a first set of features used by the processor to calculate a selection score which is indicative of presence or absence of the motor control disorder in the subject;
(b) a second algorithmic model to identify a second set of features used by the processor to calculate a severity score which is indicative of severity of the motor control disorder in the subject; and
(c) a third algorithmic model to identify a third set of features used by the processor to calculate a progression score which is indicative of progression of the motor control disorder in the subject.

67. The automated method according to claim 65, comprising the step of categorising movement dysfunction in the subject, by the processor calculating a contribution made by each of the first or second set of features to each of a plurality of movement characteristics that are attributable to movement dysfunction in the subject and optionally, wherein the plurality of movement characteristics correlate to clinically accepted descriptions movement disorder and optionally, wherein the clinically accepted descriptions relate to one or more of stability, timing, accuracy and rhythmicity of the movement.

68. The automated method according to claim 65, wherein the received movement data is obtained from a movement detection device and comprises at least one or both of:
pressure data corresponding to pressure applied to the device by the subject; and
motion data comprising one or more of position of the limb, acceleration of the limb and angular position of the limb.

69. The automated method according to claim 65, wherein the received movement data is collected while the subject performs a movement task and preferably wherein the movement task is or simulates an activity of daily living.
